(19) United States
(12) Patent Application Publication
(10) Pub. No.: US 2024/0075280 A1
(43) Pub. Date: Mar. 7, 2024

(54) ELECTRODE PLACEMENT AND SECUREMENT

(71) Applicant: COCHLEAR LIMITED, Macquarie University, NSW (AU)

(72) Inventors: Antonin RAMBAULT, Macquarie University (AU); Carl VAN HIMBEECK, Macquarie University (AU); Marcus ANDERSSON, Macquarie University (AU); Peter GIBSON, Macquarie University (AU); Nicholas Charles Kendall PAWSEY, Macquarie University (AU); Paul Michael CARTER, Macquarie University (AU)

(21) Appl. No.: 18/268,457

(22) PCT Filed: Dec. 16, 2021
(86) PCT No.: PCT/IB2021/061870

§ 371 Date: Jun. 20, 2023

Related U.S. Application Data
(60) Provisional application No. 63/126,820, filed on Dec. 17, 2020.

(51) Int. Cl. A61N 1/05

(52) U.S. Cl. CPC A61N 1/0541

(57) ABSTRACT

An apparatus, including an electrode, wherein the electrode is at least one of directly or indirectly fixed to an otic capsule or tissue associated with the otic capsule of a human at least in part with cured securement material, wherein the electrode is part of a human ailment treatment and/or mitigation system, at least in part implantable in the human.

### CROSS-REFERENCE TO RELATED APPLICATIONS

This application claims priority to U.S. Provisional Application No. 63/126,820, entitled ELECTRODE PLACEMENT AND SECUREMENT, filed on Dec. 17, 2020, naming Antonin RAMBAULT of Mechelen, Belgium as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatuses, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is an apparatus, comprising an electrode, wherein the electrode is at least one of directly or indirectly fixed to an otic capsule or tissue associated with the otic capsule of a human at least in part with cured securement material.

In an exemplary embodiment, there is a method, comprising treating a neurological ailment and/or mitigating effects of the neurological ailment of a human with an electrode by at least one of: directly or indirectly fixed to tissue of a recipient with cured electrically conductive securement material, located in a partial artificial hole in bone of a skull; or directly or indirectly fixed to tissue of a recipient with cured securement material, wherein the electrode is in electrically conductive communication with soft tissue in the head of the human.

In an exemplary embodiment, there is an apparatus, comprising an electrode, wherein the apparatus includes an arrangement that channels an uncured securement material, applied under pressure into the arrangement, to desired locations, and the apparatus is an implantable apparatus implantable in a human recipient attachable to bone of the human recipient.

In an exemplary embodiment, there is a method, comprising placing an electrode against tissue of an inner ear of a human and applying uncured securement material to bone of the human to hold the electrode against the tissue of the inner ear of the human.

### BRIEF DESCRIPTION OF THE DRAWINGS

Some embodiments are described below with reference to the attached drawings, in which:

FIG. 1 is a perspective view of the ear system of a human;

FIG. 2 is a perspective view of a tinnitus treatment system including a portion implanted in a human according to an exemplary embodiment;

FIGS. 3 and 4 present schematics of an epilepsy treatment and/or management system implanted in a human;

FIGS. 5 and 6 depict exemplary components utilizeable in some embodiments of the teachings herein;

FIGS. 7 to 13A depict exemplary features of exemplary embodiments associated with electrodes according to the teachings herein;

FIGS. 14-14B depict an exemplary arrangement for an electrode in contact with the round window;

FIGS. 15 to 20 depict an exemplary arrangement that channels securement material according to an exemplary embodiment; and

FIGS. 21 to 28 depict an additional exemplary figures of exemplary embodiments associated with electrodes according to some of the teachings herein.

### DETAILED DESCRIPTION

Embodiments of at least some teachings detailed herein can be utilized to treat or otherwise mitigate the effects of tinnitus. Embodiments include the application of an electrical signal to tissue of a recipient who suffers from tinnitus as part of the therapy to reduce or otherwise eliminate the propensity to experience a tinnitus as part of an overall treatment, whether to completely rid the recipient of tinnitus or otherwise reduce the occurrence thereof as a result of continued treatment. Here, the electrical signal is applied to tissue of the ear system to evoke a hearing percept or otherwise stimulate nerves associated with the auditory system (which may or may not evoke a hearing percept, or at least something perceived as sound) in a manner that treats the tinnitus. Embodiments also include the application of an electrical signal to tissue of the recipient who suffers from tinnitus as part of a real time tinnitus mitigation program, where the signal evoke a hearing percept or otherwise stimulates nerves (whether resulting in a hearing percept or not) associated with the auditory system in a manner that blocks or otherwise cancels the perception of tinnitus.

Any tinnitus mitigation and/or treatment regime that can utilize the teachings detailed herein should be considered part of the teachings detailed herein, and these teachings can be utilized with such where an electrode is utilized to provide electrical current to tissue of a recipient suffering from tinnitus or who may suffer from tinnitus.

Embodiments include utilizing any one or more of the teachings detailed herein in conjunction with a tinnitus treatment and/or mitigation method. Embodiments include utilizing any one or more of the teachings detailed herein in conjunction with a tinnitus treatment and/or mitigation system.

Exemplary embodiments will sometimes be described in terms of the basics of a cochlear implant, because a cochlear implant is a device that can provide electrical stimulation to tissue of the ear system of a recipient, and thus such arrangements can be utilized or otherwise modified to be utilized for the tinnitus treatment and/or mitigation techniques detailed herein. That said, it is noted that the teachings detailed herein and/or variations thereof can be utilized with other types of hearing prostheses, such as by way of example, bone conduction devices, DACI/DACS/middle ear implants, etc., where such devices are combined with electrodes for whatever reason Indeed, any disclosure herein of an electrode arrangement corresponds to an alternate disclosure of utilizing such with an apparatus that includes an actuator of a middle ear implant or a bone conduction device or a DACS/DACI, etc., and a disclosure of the alternate electronics of the implant to implement such. Still further, it is noted that the teachings detailed herein and/or variations thereof can be utilized with other types of prostheses, such as pacemakers, muscle stimulators, etc. In some instances, the teachings detailed herein and/or variations thereof are applicable to any type of implanted component that utilizes feedthroughs. In this regard, any of the aforementioned devices can be combined with the teachings detailed herein in an overall device.

To be clear, the techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, or the framework for such, such as a cochlear implant, or a tinnitus mitigation and/or treatment apparatus, corresponds to a disclosure of another embodiment of using such teaching with another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable/implanted microphones, whether or not used as part of a tinnitus mitigation and/or treatment apparatus, and/or a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. Indeed, the teachings herein can be used with specialized communication devices, such as military communication devices, factory floor communication devices, professional sports communication devices, etc.

The teachings herein can be used to treat epilepsy and/or mitigate the effects of epilepsy and/or to treat sleep apnea, where such devices utilize electrodes to sense physiologic features of the body that might be indicative of the occurrence of such and/or utilize electrodes to treat or otherwise mitigate the effects of such. Accordingly, any of the teachings detailed herein can be combined with such devices, systems, and methods providing that the art enables such, and embodiments include such. While the teachings detailed herein will be described for the most part with respect to tinnitus mitigation and/or treatment devices, utilizing as a framework hearing prosthesis technologies, in keeping with the above, it is noted that any disclosure herein with respect to a tinnitus treatment and/or mitigation prostheses and/or a hearing prosthesis, corresponds to a disclosure of another embodiment of utilizing the associated teachings with respect to any of the other prostheses noted herein, whether a species of a hearing prosthesis, or a species of a sensory prosthesis.

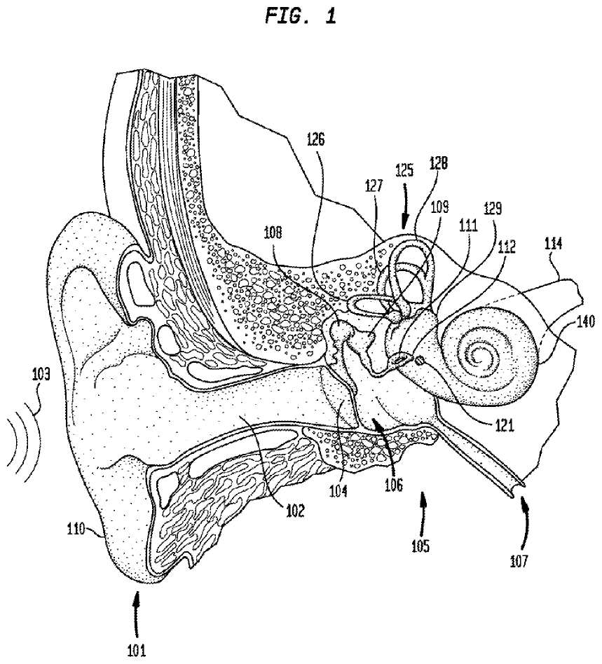

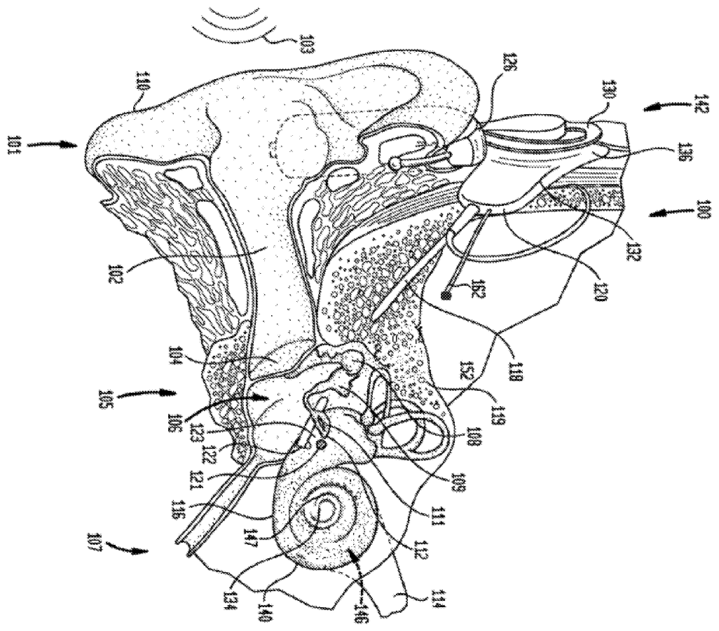

FIG. 1 is a perspective view of a human skull showing the anatomy of the human ear. As shown in FIG. 1, the human ear comprises an outer ear 101, a middle ear 105, and an inner ear 107. In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112, which is adjacent round window 121. This vibration is coupled through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to the vibration of tympanic membrane 104. This vibration sets up waves of fluid motion of the perilymph within cochlea 140. Such fluid motion, in turn, activates hair cells (not shown) inside cochlea 140. Activation of the hair cells causes nerve impulses to be generated and transferred through the spiral ganglion cells (not shown) and auditory nerve 114 to the brain (also not shown) where they cause a hearing percept.

As shown in FIG. 1, semicircular canals 125 are three half-circular, interconnected tubes located adjacent cochlea 140. Vestibule 129 provides fluid communication between semicircular canals 125 and cochlea 140. The three canals are the horizontal semicircular canal 126, the posterior semicircular canal 127, and the superior semicircular canal 128. The canals 126, 127, and 128 are aligned approximately orthogonally to one another. Specifically, horizontal canal 126 is aligned roughly horizontally in the head, while the superior 128 and posterior canals 127 are aligned roughly at a 45 degree angle to a vertical through the center of the individual's head.

Each canal is filled with a fluid called endolymph and contains a motion sensor with tiny hairs (not shown) whose ends are embedded in a gelatinous structure called the cupula (also not shown). As the orientation of the skull changes, the endolymph is forced into different sections of the canals. The hairs detect when the endolymph passes thereby, and a signal is then sent to the brain. Using these hair cells, horizontal canal 126 detects horizontal head movements, while the superior 128 and posterior 127 canals detect vertical head movements.

FIG. 2 is perspective view of a tinnitus treatment implant, referred to as implant 100, implanted in a recipient. As shown, implant 100 comprises one or more components which are temporarily or permanently implanted in the recipient. Implant 100 is shown in FIG. 2 with an external device 142 which, as described below, is configured to provide power and/or data (including control data) to the implant.

Briefly, the implant 100 can be loosely based on a cochlear implant (partially implantable or totally implantable). The arranging of implant 100 can receive power and/or data in a manner similar to and/or the same as or otherwise as modified to implement tinnitus treating and convert such to an electrical stimulation signal that is ultimately delivered to tissue. As will be detailed herein, some arrangements may or may not have an implanted processor. Any arrangement that can be used to provide electrical stimulation to an electrode to stimulate tissue to treat tinnitus (or to treat epilepsy, for that matter—more on this below) can be used in some embodiments.

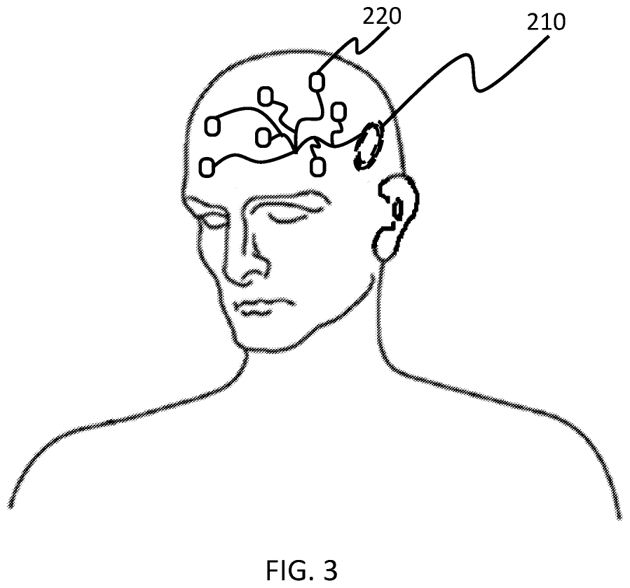

In the illustrative arrangement of FIG. 2, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. As would be appreciated, various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 3 is merely illustrative, and other external devices may be used with embodiments of the present invention.

Implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Implant 100 further comprises a main implantable component 120 and an elongate electrode assembly 118. In embodiments, internal energy transfer assembly 132 and main implantable component 120 are hermetically sealed within a biocompatible housing. In embodiments of the present invention, main implantable component 120 can include but also may not include a processing unit (not shown), which can be a processor, to convert energy and/or data received by the implant into data and/or energy signals. Main implantable component 120 further includes a stimulator unit (also not shown) which generates electrical stimulation signals based on the data signals. The electrical stimulation signals are delivered to the recipient via elongate electrode assembly 118. In some embodiments, the main implantable component 120 has the stimulator unit but little to no processing capability. In an exemplary embodiment, the implant is configured such that the stimulator unit receives a signal, energy and/or data, from the implanted antenna (which receives a signal, energy and/or data, transcutaneous from the external coil) and converts the signal to an electrical output/an electrical stimulation signal which is provided to the elongate electrode assembly 118.

The above said, in an exemplary embodiment, the implant 100 is a totally implantable apparatus that includes a power source (e.g., battery), and is configured to operate in a manner akin to a totally implantable hearing prosthesis, as modified for tinnitus treatment.

Elongate electrode assembly 118 has a proximal end connected to main implantable component 120, and a distal end that includes an electrode that is located abutting the cochlea 140. In the embodiment shown in FIG. 2, the electrode (not shown) is located in drilled partial hole 122. Electrode assembly 118 extends from main implantable component 120 to cochlea 140 through mastoid bone 119, to the otic capsule. The teachings below will frequently relate to the “working end” of the elongate electrode assembly 118. By way of example only and not by way of limitation, in an exemplary embodiment, any one or more of the teachings below relating to an electrode and the associated disclosure there with correspond to the working end of the electrode assembly 118 of FIG. 2. In exemplary embodiments, there will often be disclosed a lead assembly including electrically conductive wires and insulative material surrounding those wires extending from the electrode. The insulative material and the electrically conductive wires correspond in effect to the electrode assembly 118 seen in FIG. 2. That said, other embodiments can utilize other configurations, and the teachings detailed below with respect to the electrode and the associated components can be applied in other types of systems, such as a system where there is no stimulator unit, and instead where the electrical stimulation flows directly or indirectly from the inductance coil, albeit through potentially a receiver unit with respect to indirect flow.

As noted, implant 100 can comprise a totally implantable prosthesis that is capable of operating, at least for a period of time, without the need for external device 142. Therefore, implant 100 further comprises a rechargeable power source (not shown) that stores power received from external device 142. The power source may comprise, for example, a rechargeable battery. Alternatively, a long term non-rechargeable power source that is implanted and remains implanted may be used. During operation of implant 100, the power stored by the power source can be distributed to the various other implanted components as needed. The power source may be located in main implantable component 120, or disposed in a separate implanted location.

As seen, there is a return electrode/reference electrode at the end of lead 162. This provides a return/reference for the electrode at the end of the electrode assembly 118.

Another exemplary arrangement that can use the electrode technologies detailed here is an epilepsy monitoring and/or treatment device. FIG. 3 provides an exemplary embodiment of an EEG system (that can be an epilepsy monitoring system) that is implanted in the recipient, where read/sense electrodes 220 are arrayed inside a recipient's head and in signal communication with a coil 210 via electrical leads. In this embodiment, the implanted device has no recording/storage capabilities, and requires an external device to receive a signal from the implanted inductance coil 210 so as to retrieve in real time the signal therefrom. Not shown is an implantable component that converts the electricity sensed by the sensor/read electrodes into a signal that is transmitted by the inductance coil. In an exemplary embodiment, the sensor arrangement seen in FIG. 3 is an implanted EEG sensor arrangement.

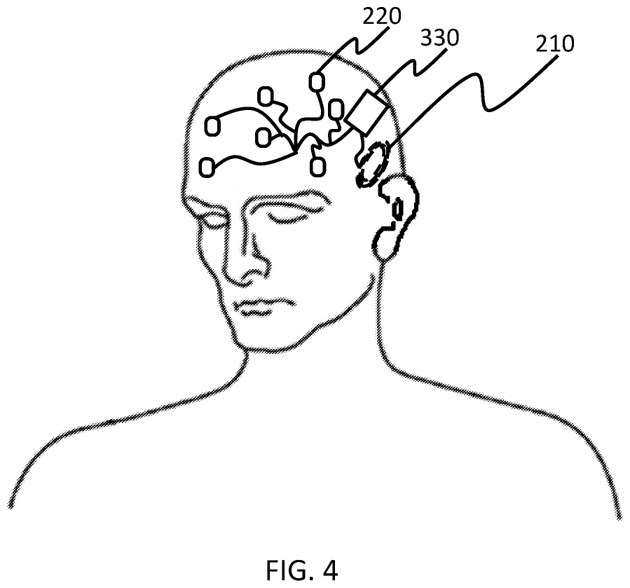

FIG. 4 depicts another arrangement of an implantable sensor arrangement that again includes the sensor/read electrodes 220 and the leads. Here, in this embodiment, there is a housing 330 which includes circuitry that is configured to receive the signals from the leads from the electrodes 220 and record the data therefrom or otherwise store the data, and permits the data to be periodically read from an external device when the external device comes into signal communication with the implanted inductance coil 210. Alternatively, and/or in addition to this, the circuitry is configured to periodically energize the inductance coil 210 so as to provide the data to the coil 210 so that it creates an inductance signal which in turn communicates with an external component that reads the signal and thus reads the data associated with the electrodes. Thus, in at least some exemplary embodiments, the implantable apparatus is configured to stream the data. Still further, in some embodiments, the data is not streamed, but instead provided in bursts.

Any arrangement that can enable the data associated with the read electrodes to be provided from inside the recipient to outside the recipient can be utilized in at least some exemplary embodiments. In this regard, traditional implanted EEG sensor arrangements can be obtained and modified so as to implement the teachings detailed herein and/or variations thereof.

It is noted that some embodiments of the sensor arrangement of FIG. 4 includes an implanted battery or otherwise implanted power storage arrangement, while in other embodiments the arrangement specifically does not, making the arrangement akin to the embodiment of FIG. 3.

It is noted that while the above is presented in terms of a monitoring system, the teachings above can also be representative of an epilepsy monitoring (e.g., seizure) treatment arrangement where the electrodes are used to provide electrical stimulation to the skull, and thus to the brain. More on this below.

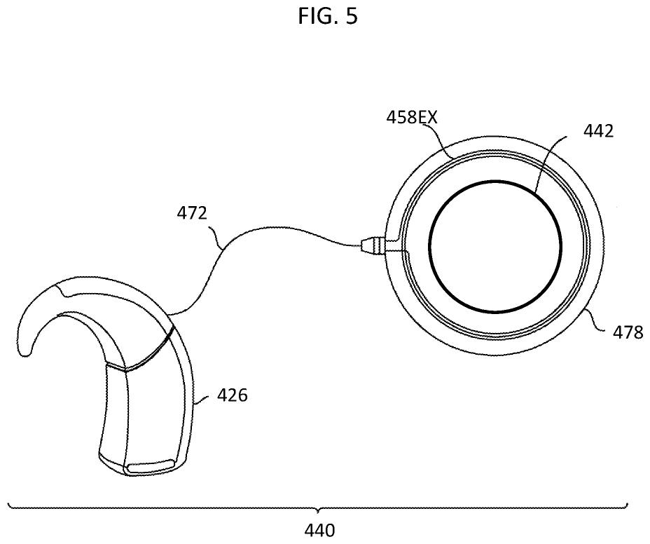

It is noted that while the embodiments detailed herein are described in terms of utilizing an external device that is fixed or otherwise relatively immobile to communicate and/or power the implanted component, it is to be understood that these devices can also be powered by their traditional external components. In this regard, FIG. 5 depicts an exemplary external component 440. External component 440 can correspond to external component of FIG. 2 above. As can be seen, external component 440 includes a behind-the-ear (BTE) device 426 which is connected via cable 472 to an exemplary headpiece 478 including an external inductance coil 458EX, corresponding to the external coil of FIG. 2. As illustrated, the external component 440 comprises the headpiece 478 that includes the coil 458EX and a magnet 442. This magnet 442 interacts with the implanted magnet (or implanted magnetic material) of the implantable component to hold the headpiece 478 against the skin of the recipient. In an exemplary embodiment, the external component 440 is configured to transmit and/or receive magnetic data and/or transmit power transcutaneously via coil 458EX to the implantable component, which includes an inductance coil. The coil 458X is electrically coupled to BTE device 426 via cable 472. BTE device 426 may include, for example, at least some of the components of the external devices/components described herein.

Accordingly, in an exemplary embodiment, external component 440 can be utilized with the implantable component that is an implantable tinnitus treatment apparatus and/or an epilepsy treatment/monitoring implant as detailed herein where the implanted coil is implanted near or in the head. In this regard, the external device of FIG. 5 can be utilized in combination with the exemplary EEG system of FIGS. 3 and 4 (or the tinnitus treatment implant of FIG. 2 and/or other arrangements herein). Indeed, in an exemplary embodiment where, for example, the implanted coil of the EKG system detailed herein is located in the upper reaches of the torso, such as at the top of the chest, it is possible to utilize the external device 440 with such a system by snaking the lead 472 downward through a person's shirt collar or the like to the person's chest or shoulder. That said, in alternate embodiments, a specialized external device especially for the EKG system can be utilized, where, for example, the non-coil portions (e.g., the equivalent of the BTE component 426) is worn on a chain around the person's neck like a pendant, and the coil is magnetically adhered to the coil inside the person. Further, an off-the-ear (OTE) device could be used, which can be a single unit located over the coil, wherever such is located. This device would not be on a pendant, but instead could be held by a magnet, etc., to the recipient.

In an exemplary embodiment, with respect to the electrodes depicted in the figures just describe, as will be detailed below, in some embodiments, the location of the electrodes correspond to partial holes and/or excavations in the skull where the electrodes are located. The teachings detailed below with respect to the electrodes can correspond to the electrodes detailed in FIGS. 3 and 4.

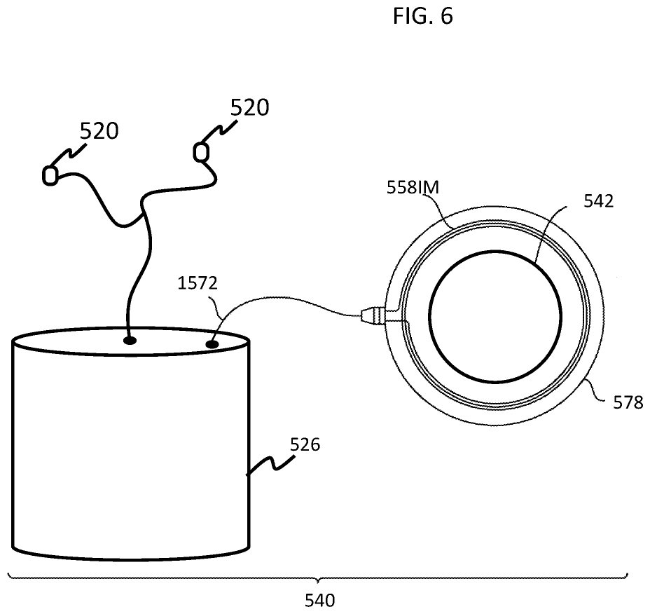

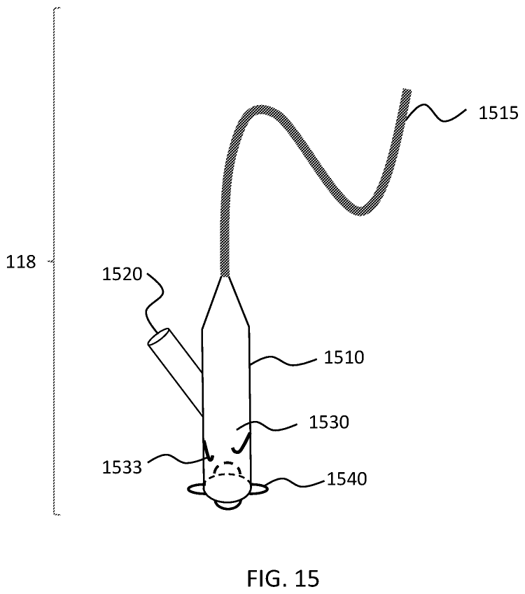

With respect to the implantable device, FIG. 6 provides an exemplary functional arrangement of an implantable device 540 that is configured to transcutaneously communicate via an inductance field with the external device of FIG. 5 or an analogous device. Implantable component 540 can correspond to a tinnitus treatment apparatus or an epilepsy treatment apparatus. Alternatively, and/or in addition to this, the implantable component of FIG. 6 can correspond by way of representation to the implantable component of the EEG embodiment or the EKG embodiment or the retinal implant embodiment. As can be seen, external component 540 includes an implantable housing 526 which is connected via cable 572 to an exemplary implanted coil apparatus 578 including an implanted inductance coil 558IM, corresponding to the external coil of FIG. 1 in this exemplary embodiment, where FIG. 15 represents a tinnitus treatment implant. As illustrated, the implantable component 540 comprises an implanted inductance communication assembly that includes the coil 558IM and a magnet 542. This magnet 152 interacts with the external magnet of the implantable component to hold the headpiece 478 against the skin of the recipient. In an exemplary embodiment, the implantable component 540 is configured to transmit and/or receive magnetic data and/or receive power transcutaneously via coil 558IM from the external component, which includes an inductance coil as detailed above. The coil 558IM is electrically coupled to the housing 526 via cable 572. The housing 526 may include may include, for example, at least some of the components of the implantable components herein, such as for example, the stimulator of the implant 100 (which can be a modified and/or a pure cochlear implant stimulator (where the full capability thereof is not used) where the embodiment of FIG. 6 represents such.

Housing 526/the apparatus thereof, can correspond to element 320 or the main body 120 above. Element 540 can correspond to implant 100 above.

Implantable component 540 also includes a stimulating assembly which includes leads extending from the housing 526 that ultimately extend to electrodes 520, as seen. In the embodiment where FIG. 6 represents the implantable component of the cochlear implant, electrodes 520 and the associated leads functionally represents the electrode assembly of a cochlear implant, although it is specifically noted that in a real cochlear implant, electrodes 520 would be supported by a carrier member instead of being “free” as shown. That said, in an exemplary embodiment, FIG. 6 can represent the EEG and/or the EKG systems detailed above, where the electrodes 1520 are read/sense electrodes. Still further, in an exemplary embodiment, the implantable component of FIG. 6 can represent the retinal implant. Note further, that in an exemplary embodiment, the electrodes 520 are replaced with mechanical actuators, and thus the embodiment of FIG. 6 represents an active transcutaneous bone conduction device and/or a middle ear implant, etc.

In this regard, FIG. 6 is presented for conceptual purposes to represent how the external component of FIG. 5 communicates with the implanted component. Along these lines, in an exemplary embodiment, the external component's magnet magnetically aligns with the implantable component's magnet, thus aligning the external coil with the implanted coil. This can have utilitarian value as aligning the coils provide efficiency relative to that which would be the case if the coils are misaligned. By way of example only and not by way of limitation, in an exemplary embodiment, the magnets are disk magnets having the north-south polarity aligned with the axis of rotation of the disks. In this regard, the magnets want to align the magnetic fields with one another, and thus by holding the respective coils at predetermined and control distances from the respective magnets utilizing the structure of the external component and/or the implantable components (e.g., a silicone body) the coils will become aligned with each other because the magnets will become aligned with each other.

Embodiments are directed to securing electrode contact against tissue, such as bone, or a round window of the cochlea, etc. Embodiments include electrode(s) utilized to treat tinnitus, such as via the application of electrical current to the ear system of a person suffering from tinnitus. By way of example only and not by way of limitation, in an exemplary embodiment, one or more electrodes are placed against or otherwise in electrical communication with the tissue (e.g., bone) of the otic capsule/bony labyrinth, etc. In an exemplary embodiment, by way of example, one or more electrodes are placed against or otherwise in electrical communication with the round window of the cochlea. The concept here is that the electrical current supplied by the electrode or otherwise conducted from the electrodes to the tissue, will stimulate the inner ear nerves or otherwise the auditory nervous system, in a manner that can be utilized to treat and/or otherwise mitigate the effects of tinnitus.

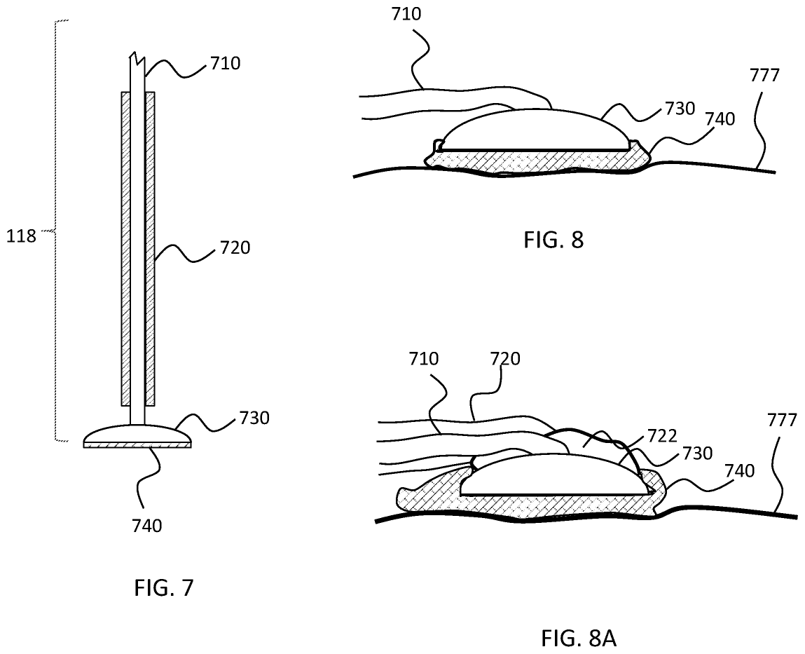

FIG. 7 presents an exemplary embodiment of an electrode design they can have utilitarian value with respect to embodiments according to the teachings detailed herein. As seen, there is an assembly 118, which can correspond to that detailed above with respect to FIG. 2, which is presented here as including the electrode 730. More specifically, there is a conductive member 710, which can be an electrical lead, which is housed in an electrically insulative sheath 720, which can be made of any biocompatible material that has a high impedance or otherwise that can provide electrical insulation from body fluids or otherwise the ambient environment. FIG. 7 shows the sheath extending toward but not all the way to the electrode 730. In an exemplary embodiment, the sheath can extend all the way to the electrode, and in some embodiments can extend to the sides of the electrode in some embodiments. Or another component separate from the sheath which also has high impedance and otherwise provides sufficient electrical insulation can be utilized. In an exemplary embodiment, a cap made out of a high impedance material can be located at the end of the sheath 720, and the electrode 730 can be placed inside that cap.

In this exemplary embodiment, the electrode 730 has a circular or oval or otherwise curved surface facing downwards with respect to the orientation of FIG. 7. In an exemplary embodiment, slightly different than that shown in FIG. 7, the electrode 730 is a flat disk welded or otherwise electrically connected and/or mechanically connected to the lead 710. In this exemplary embodiment, the faces of the disk would both be flat on both sides (facing downward and facing upward respectively), and the side/lateral services would be curved (which would be a cylindrical body if the electrode extended sufficiently in the high direction). In this exemplary embodiment, the back of the electrode is domed, and is thus not a proper disk per se. Any shape configuration that can enable the electrode to be attached to tissue of the recipient and otherwise enable the conduction of electricity/electrical current to the tissue can be utilized in at least some exemplary embodiments.

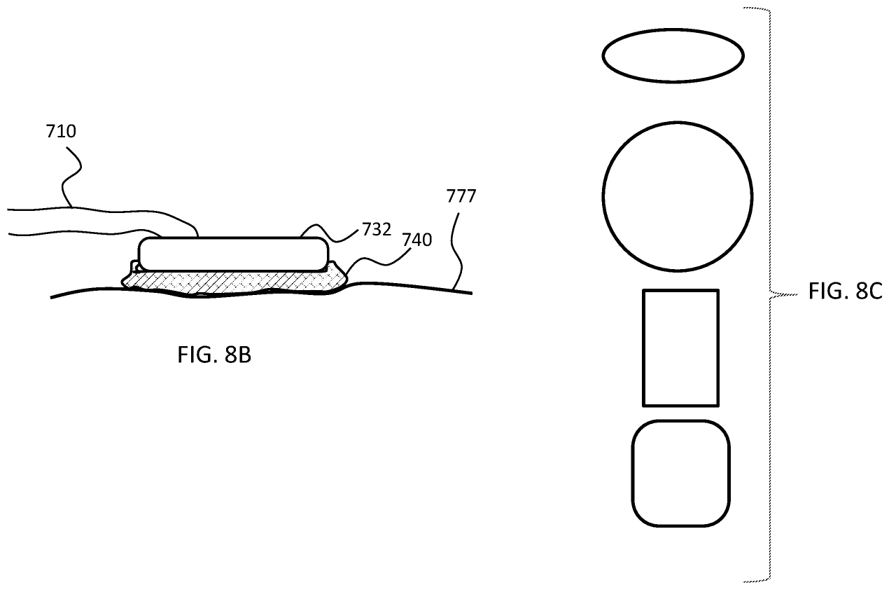

Also as seen in FIG. 7 is the utilization of bone cement 740. In this exemplary embodiment, bone cement is conductive, at least in a manner that is sufficient to enable sufficient amounts of current to travel from the electrode into the tissue to which the electrode 730 is to transfer current there to. Sometimes herein it is stated that the electrode is in contact with the tissue. In this exemplary embodiment, as further seen in FIG. 8, the electrode 730 is in indirect contact with the tissue to which electrical current is to be provided, here, bone 777. More particularly, FIG. 8 shows an exemplary scenario where the electrode 730 is indirectly attached to bone 777 by conductive cement 740.

In an exemplary embodiment, the bone cement that is utilized has a conductivity at least about 50% of that of bone, such as the skull bone in general, and the jawbone and/or the mastoid bone in particular. In an exemplary embodiment, this is on a per unit volume basis. Additional details will be provided below. But briefly, in an exemplary embodiment, the bone cement is at least or equal to 30, 35, 40, 45, 50, 55, 60, 65, 70, 75, 80, 85, 90, 95, 100, 110, 120, 130, 140, 150, 160 170, 180, 190, 200, 250, 300, 350, 400, 450, or 500 percent or more, or any value or range of values therebetween in 1% increments (e.g., 67, 122, 53 to 97 percent, etc.), inclusive, as conductive as the jawbone and/or mastoid bone and/or a femur, etc., and can be on a per unit volume basis.

FIG. 8A depicts another exemplary embodiment of the utilization of the electrode teachings, where here, the 722 can be seen, which interfaces with the insulative sheath 720. Here, as can be seen, the bone cement 740 extends around the sides of the electrode 730 (more so than the arrangement shown in FIG. 8). This can have utilitarian value with respect to increasing the surface area of contact between the bone cement and the electrode, and also the amount of contact/the surface area contact between the bone cement and the bone.

It is briefly noted that as can be inferred from the FIGS. 7 to 8A, the lead portion is flexible or otherwise can be moved relative to the electrode portion. That said, in alternate embodiments, at least a portion of the lead portion proximate the electrode could be rigid relative to the electrode and otherwise is not movable relative to the electrode.

It is briefly noted that any disclosure herein of bone cement corresponds to an alternate disclosure of any other system that can enable adhesion they can have utilitarian value or otherwise be utilized in accordance with the teachings detailed herein. By way of example only and not by way of limitation, biocompatible conductive adhesives can be utilized alternatively and/or in instead of the biocompatible bone cement. It is also noted that bone cement as used herein can also be utilized to attach the electrode to tissue that may not necessarily be bone. Accordingly, the utilization of bone cement does not require that the electrode be in contact with bone unless otherwise stated. The tissue could be any other type of tissue where bone cement can be utilized to hold the electrode into contact therewith. Some additional examples/embodiments of cements and alternates for use are described below.

In some embodiments, hydroxyapatite based cements can be used.

Any disclosure of holding electrode into contact with tissue response to a disclosure of holding the electrode into electrical communication with that tissue unless otherwise specified.

In an exemplary embodiment, the edge(s) of the electrode can be curved inside to reduce or otherwise eliminate what might otherwise damage the bone during the electrode placement. FIG. 8B depicts an exemplary embodiment where a disk shaped electrode 732 having such rounded edges is utilized.

FIG. 8C depicts bottom views (e.g., looking up from the bone 777) and/or top views of some exemplary electrodes that can be utilized in some embodiments

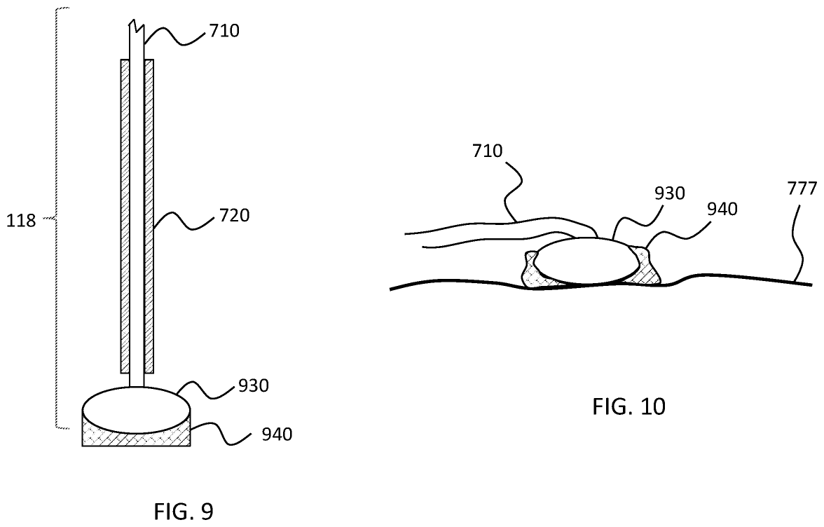

FIG. 9 depicts another exemplary embodiment of an electrode, electrode 930, along with bone cement 940. Here, at least the bottom of the electrode 930 is a dome shaped body. In an exemplary embodiment, the electrode is a half ball or a ball. Here, the bone cement 940 is placed on the dome shape.

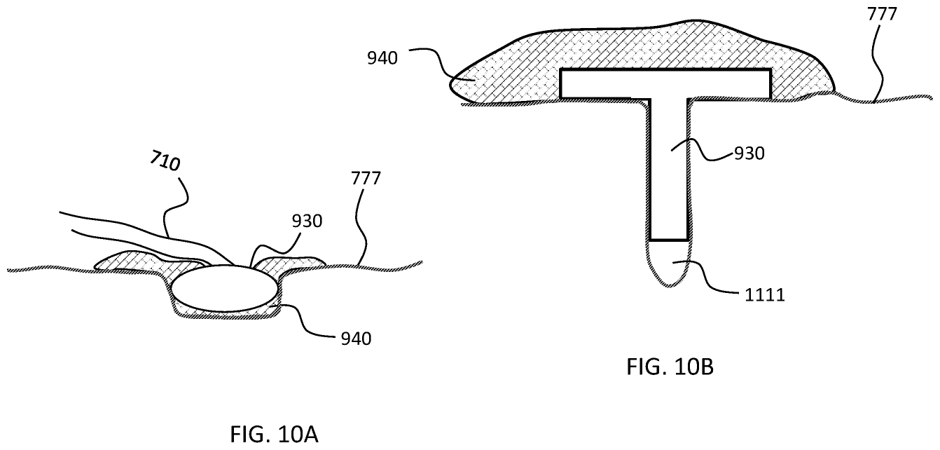

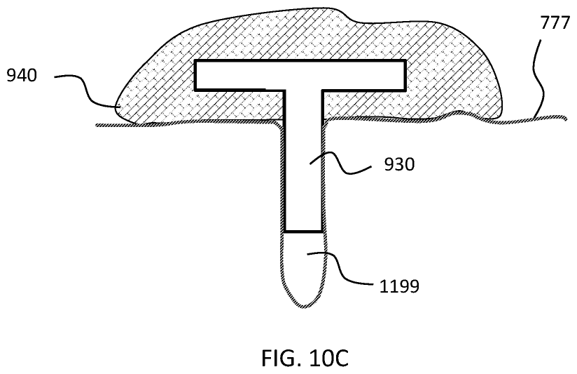

In the embodiment depicted in FIG. 10, the domed shape electrode is secured to bone 777 via a glob of bone cement 940 as shown. In this exemplary embodiment, the electrode 930 is in direct contact with the surface of the bone 777. This arrangement utilizes the sloping surface of the bottom of the electrode array to provide an area that provides direct contact with the bone and also provides a surrounding area for placement of the bone cement.

FIG. 10A depicts how the dome shape arrangement can be utilized to facilitate insertion into a hole into bone 777. The hole can be drilled into bone (it is a partial hole/not a through hole) and can be sized to be about proximate the outer diameter of the electrode. In this embodiment, the electrode can be snug fitted or interference fitted or slip fitted or closely fitted into the hole. This can provide for direct contact with the electrode and the bone. That said, there may not necessarily be direct contact. Here, there is direct contact around the periphery of the electrode but not the bottom of the electrode. In an alternate embodiment, direct contact between the electrode and the bottom of the hole can be achieve. Here, the bone cement 940 is located underneath the electrode 930 and on top of the electrode 930. This provides additional area for the adhesion properties for the electrode 930. This also provides expanded area for electrical conductivity with respect to electricity flow from the electrode into the bone cement and then into bone.

In another exemplary embodiment, the electrode can be a T shaped body (T shaped cross-section) assembly or the like, such as that shown in FIG. 10B. Here, there is a hole that is drilled into the bone 777, again not a through hole, where the leg of the T is slip fit or interference fitted or snug fitted or clearance fitted into the hole (shown is a snug fit) where the top of the T, or more specifically, the bottom surface of the top of the T, contacts the bone directly (just as does the leg of the T). The bone cement 940 is applied in a manner where the bone cement adheres to the surface of the bone 777 outside the periphery of the top of the electrode 930. (Not shown is the lead extending from the electrode to the stimulator device—in an exemplary embodiment, the bone cement 940 would be packed around that lead/the sheath of the lead). In this embodiment, the bone cement need not necessarily be conductive. In this regard, the bone cement can have a high impedance, or relatively high impedance, that will effectively or substantially or utilitarianly insulate the electrode from the ambient environment. In an exemplary embodiment, the impedance of the bone cement could be more than 100, 200, 300, 400, 500, 600, 700, 800, 900, 1000, 2000, 3000, 4000, 5000, 10,000, 20,00, 50,000, 100,000 percent of the impedance of bone (jaw bone or mastoid bone, in some embodiments) or any value or range of values therebetween in 10 increments, such as, for example, on a per unit volume basis. In such embodiments, it is the direct contact of the electrode that is relied upon for the transfer of electricity into the bone. That said, in an alternate embodiment, the bone cement 940 utilized in FIG. 10B can be the conductive bone cement. In some embodiments, the conductivity of the bone cement relative to the ambient environment is not a concern, and the surface area that is exposed to the ambient environment, while large, is not exposed to a conductive environment. That said, in an exemplary embodiment, this surface area that is exposed is significantly large enough that it could be problematic or otherwise could result in other phenomena that are not utilitarian. In an exemplary embodiment, an insulative coding can be placed over the bone cement, which could be a cap extending from one side of the bone cement to the other side of the bone cement, and contacting the bone. Indeed, nonconductive bone cement can be utilized to place the cap against the surface of the bone 777. That said, in an exemplary embodiment, a nonconductive material can be coated over the top, extending over past the sides of the bone cement on to the bone.

FIG. 10C presents another exemplary embodiment where bone cement is utilized or otherwise is positioned below the bottom of the top of the T. again, the bone cement 940 can be conductive or nonconductive. In the embodiments of FIGS. 10B and 10C, there is a space 1111 and 1199, respectively, in an exemplary embodiment, this space can be filled beforehand with bone cement or some other adhesive. In some embodiments, the bone cement can be conductive, while in other embodiments the bone cement is not conductive.

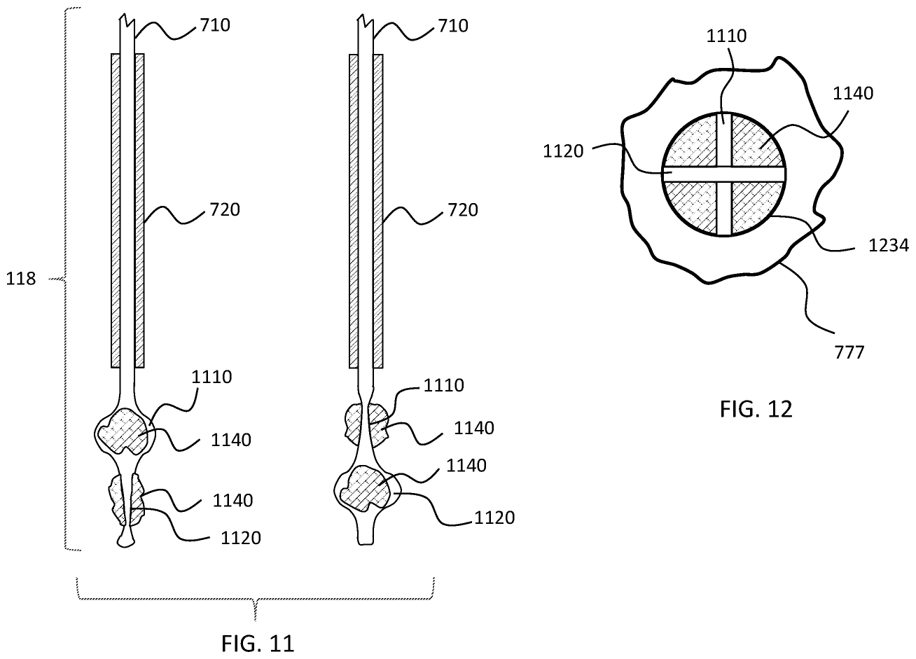

FIG. 11 depicts another exemplary embodiment that can be utilized to provide utilitarian value with respect to electrical coupling between the electrode and tissue of a human being. Here, this can be described effectively by evaluating the manufacturing process (a manufacturing process) they can be utilized to produce this arrangement. Here, the electrode is part of the body of the lead 710, or otherwise is a monolithic of the lead 710 or whatever one would call not component. Indeed, in a sense, the exposed portions of element 710 can constitute an electrode (note that the sheath 720 is depicted as ending in an arbitrary manner—the sheath would extend upwards to the stimulator for example, and would extend downward more to a utilitarian location—the portion of the “lead” 710 extending past/out of the sheath 720 would be considered the electrode in at least some exemplary scenarios). This lead 710 can be of circular cross-section. The part extending from the sheath can initially be a cylindrical body, albeit potentially slightly flexible. In an exemplary embodiment, a first segment of the lead is crimped (smushed) so that it flattens out as shown in FIG. 11 with respect to the area 1110. The area 1110 would be flat on either side, or closer to flat than rounded. A second segment of the lead is also crimped, but at an angle, such as at a 90° angle relative to the first area, this second area being area 1120 shown in FIG. 11. As shown, globs of bone cement 1140 can be located on these areas. The portion on the right side of FIG. 11 depicts a view of the portion shown on the left side, rotated about the longitudinal axis, 90 degrees. The “crimped” sections can be more than 2. Any number of crimped sections (including 1) can be used, if such is utilitarian.

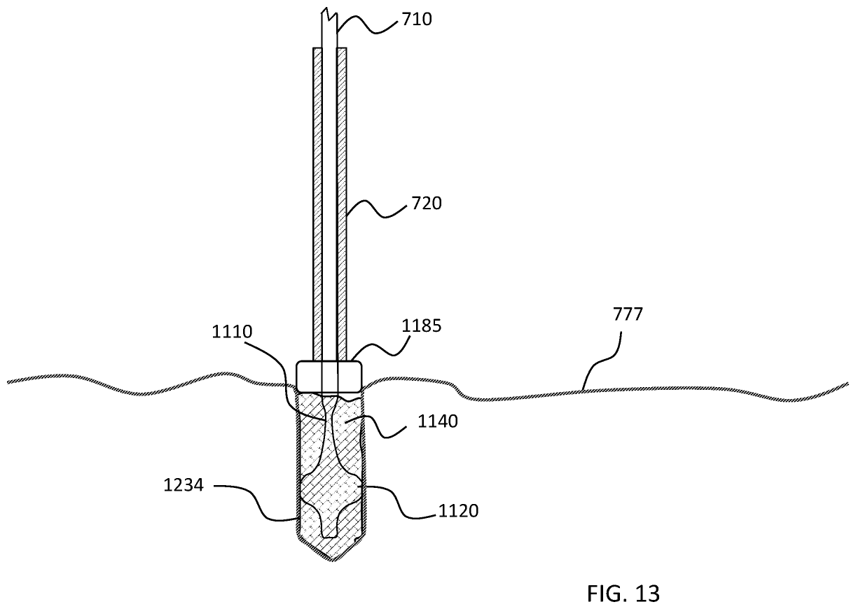

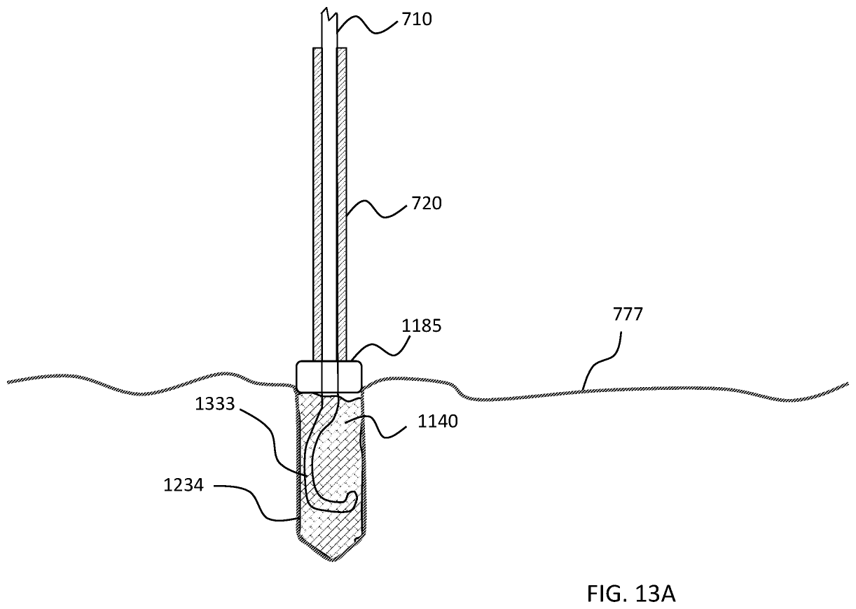

FIG. 13 shows the crimped sections in a hole 1234 in bone 777. This hole can be drilled (it is not a through hole). FIG. 12 shows the crimped sections in the hole 1234 in bone 777 (looking upward from in the hole). As can be seen, bone cement 1140 almost completely fills the hole 1120. This has the effect of locking in the crimped sections into the hole, and thus locking the electrode. Again, an insulative material can be located above the cement. As seen in FIG. 13, a plug 1185, which can be an insulative silicone body having a round cross-section, insulates the cement 1140 and/or the electrode and/or the lead from the ambient environment above he hole 1234 (the ambient environment may be the middle ear).

Different relative angles between the crimped sections can be used. While the embodiment shown herein utilizes a 90° or close to a 90° angle, the angles can be 45° or 60° or 30° or 120 degrees, etc., or any value or range of values therebetween in 1° increments, or any annular all set that can have utilitarian value.

FIG. 13A depicts another exemplary embodiment where the lead 710 is crimped and smushed and then bent into a hook like arrangement 1333 as seen, where the bone cement 1140 fills the hole 1234. That said, in an exemplary embodiment, the crimping and the smudging may not necessarily be needed. Instead, the lead 710 can simply be bent to form a hook like body while maintaining the overall circular or semicircular cross-section (there would be no flats—in the embodiment of FIG. 13A, there are flats owing to the crimping).

In an exemplary embodiment, the lead 710 can be twisted or otherwise turned into a semi-knot body, or a loose knot, where the knot would be placed in the hole 1234, and it bone cement would be utilized to fill the hole 1234 to lock the electrode therein.

These arrangements also enable the method of implantation or otherwise attachment to the bone or other tissue to be potentially simplified. In an exemplary embodiment, a drop or a blob of bone cement can be placed on the “hook” or the knot(s) or the flats, etc., and the bone cement can be transported with the electrode to the hole. Some additional bone cement might need to be added, while in an alternative embodiment, because of the nature of the cement, the “glob” of cement could be large enough to complete the entire method. Indeed, in an exemplary embodiment, bone cement might need to be removed because too much would be present. The point of this arrangement with respect to placing the bone cement on the electrode prior to moving the electrode to the recipient would be utilized to simplify or otherwise improve the process. If such is deemed not to necessarily result in an improvement in the method, the bone cement can be added after the electrode is placed into the hole or the like. Indeed, in an exemplary embodiment, a syringe can be utilized to flow the bone cement into the hole and/or to the areas around depending on the arrangement. This syringe could also be utilized to place the bone cements over the electrode array with respect to other embodiments detailed above.

The scenario where the bone cement is pre-applied to the electrode could also be utilized with the electrode(s) of FIG. 7. Indeed, in general terms, FIG. 7 shows the bone cement 740 pre-applied to the electrode 730 (albeit in a very “perfect” manner).

In some embodiments, the amount of bone cement can be applied in a globular manner so that when the electrode is placed against the bone surface, and a downward force is applied, the bone cement which spread out from underneath the outer periphery of the electrode. In an exemplary embodiment, the surgeon could utilize his or her finger and/or a tool as a spatula or the like to form/smooth the excess bone cement around the outer periphery and at least partially above the electrode.

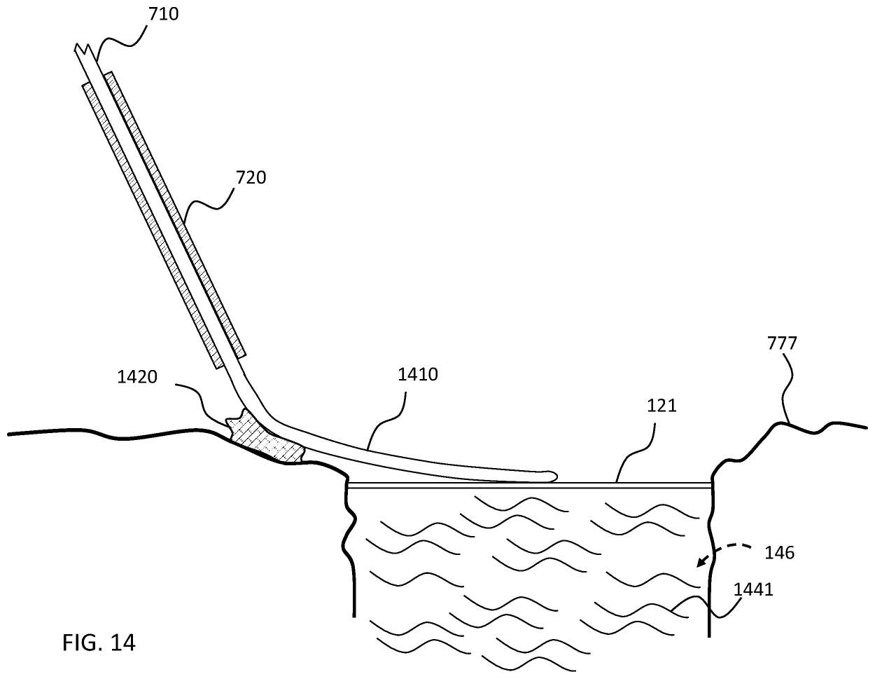

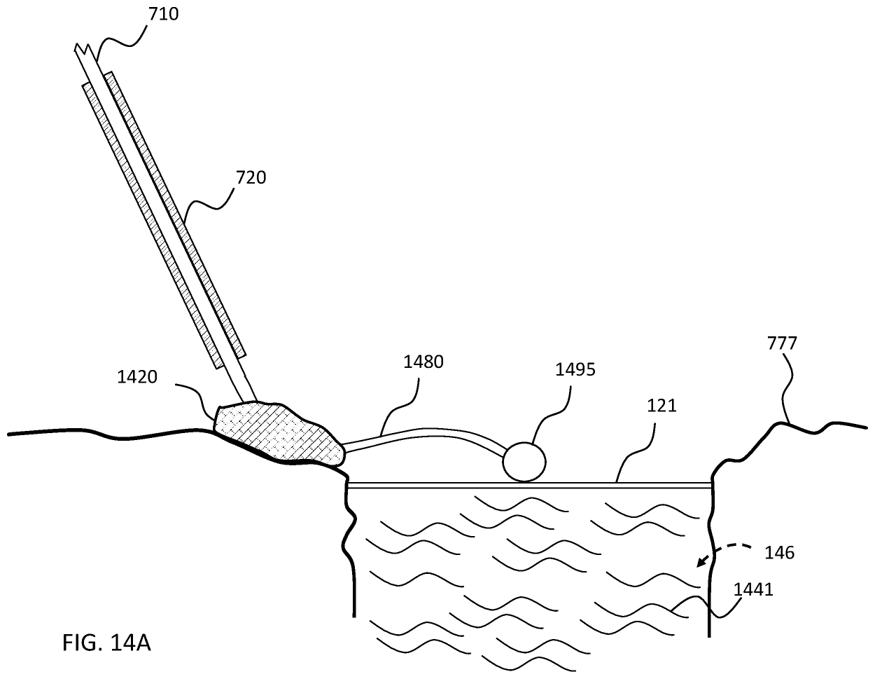

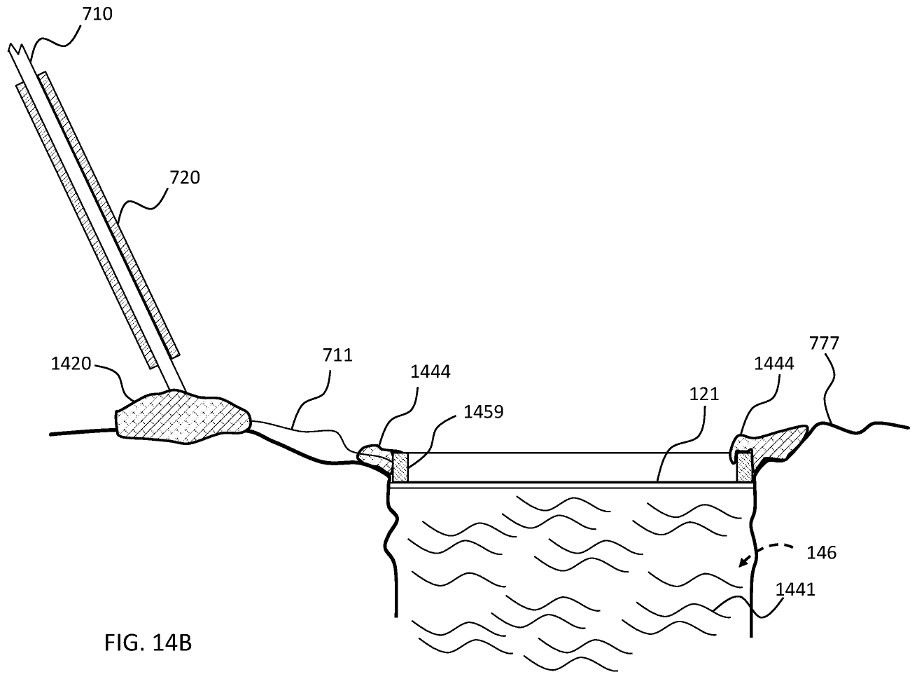

In an exemplary embodiment, there is a different target location relative to those detailed above with respect to treating tinnitus. In an exemplary embodiment, there is the application of an electrode in direct or indirect contact with a window of the cochlea, such as the round window. FIG. 14 depicts an exemplary embodiment of such an arrangement. As shown, there is round window 121 located in the round window niche in the bone 777. As shown, the inside of the cochlea 146 is filled or otherwise contains perilymph 1441. In this exemplary embodiment, the electrode 1410 is like a spatula or otherwise like a leaf spring, and is springing pressed slightly or more than slightly against the outside of the round window 121 such that there is sufficient contact area between the electrode 1410 and the outer surface the round window 121. The downward force under pressure established by the electrode 1410 will still enable the round window to function in a utilitarian manner or otherwise function so that the recipient can hear or at least perceive sounds in a manner that is no different than that which would otherwise be the case, or to the extent that there is a difference, the difference is minor and otherwise tolerable relative to the utilitarian value with respect to the arrangement when viewed in terms of balancing trade-offs.

In this exemplary embodiment, the electrode 1410 is positioned such that it has a modicum of preload and otherwise ensures utilitarian contact with the membrane. As seen, the fixation of the electrode can be established utilizing bone cement 1420 which cements the electrode 1410 at the knee point to the round window niche bone. That said, in an alternate embodiment, the cement can be applied at another location. Any location that can enable the teachings detailed herein can be utilized in at least some exemplary embodiments. Also, while the embodiment shown in FIG. 14 depicts a spatula type arrangement where the electrode is flat in and out of the page, another arrangement can be utilized where a ball electrode is mounted at the end of a cylindrical body that is preloaded so that the electrode is pushed against the round window, as seen in FIG. 14A. Here, a flexible “beam” like structure or “boom” structure 1480 extends from the glob of bone cement 1420. At the end of this flexible beam or boom structure 1480 is a ball electrode 1495. This ball electrode is pressed against the outside of the round window 121 is shown. Insulation can be utilized to cover structure 1480 and/or at least a portion of the ball electrode 1495. The preload of the structure 1480 can be such that the electrode 1495 is pressed against the round window in a manner sufficient to enable electrical flow from the electrode, through the window 121, into the perilymph 1441.

It is noted that in an exemplary embodiment, the preload is sufficient so that the direct contact between the electrode and the window is maintained throughout most if not all or at least some of the range of motions of the round window during normal or close to normal sound environments. In this regard, the range of motions of the round window will be such that the round window will move a certain amount when a recipient is exposed to certain volumes of sounds (sounds at certain decibel levels). It could be that some sounds are sufficiently high with respect to the decibel levels that the deflection of the round window may be greater than that which can be compensated for with respect to the spring forces or flexibility features of the arrangement. In an exemplary embodiment, there can be a sensor or the like that will automatically deactivate the tinnitus mechanism, or at least prevent electrical signals from being sent to the electrode, in the presence of loud sounds that cause high deflection of the round window. That said, in alternative embodiments, irrespective of the temporary lack of contact between the electrode and the round window, the current may still be supplied to the electrode.

In another exemplary embodiment, a helical spring is utilized to maintain contact between an electrode and a round window. The helical spring can be utilized as a semi-cantilevered beam arrangement—effectively replace the beam shown in the figures with a spring that happens to be helical. The helical spring can also be utilized in its traditional manner where the spring provides a downward force because the spring is slightly compressed.

Because the electrode 1410 is in direct contact with the round window membrane, there is utilitarian electrical contact between those two components, and thus providing that a sufficient voltage and/or current is utilize, utilitarian electrical conduction to the perilymph 1441. In this regard, electricity flows from the electrode 1410, into and through the round window membrane 121, and then into the perilymph 1441, and then ultimately to the nerves of the inner ear, which then are stimulated in a manner that is utilitarian with respect to treating tinnitus. In this regard, instead of the electricity traveling through bone to reach the nerve (at least with respect to the location proximate to or otherwise relative to the electrode), the electricity travels through the relatively thin membrane of the round window and then into the perilymph.

The above said, in some alternate embodiments, there is no pre-compression or the like. Instead, the electrode is adhesively connected/retained against/to the round window 121, and a very flexible lead and/or a lead that is free to move with movement of the electrode is used. In this manner, the adhesive maintains the electrical connection between the electrode and the round window. This adhesive can be between the electrode and the round window, or can be located to the side of the electrode and the round window (such as might be the case where nonconductive cement is utilized).

More specifically, FIG. 14B depicts an exemplary embodiment where a ring electrode 1459 is utilized. The ring electrode 1459 is placed at the outer periphery of the round window 121 is can be seen. This is utilitarian value with respect to the fact that the electrode contacts the areas of the round window that do not deflect as much (the center deflects more). It is noted that this technique can be utilized with respect to the other embodiments detailed herein, such as the spatula electrode and/or the ball electrode and/or the electrode held in place with a helical spring. The ideas that the contact area can be located at the periphery of the round window so that any deflection that would be frustrated by the electrode would be lower relative to that which would be the case if the electrode was at the center of the membrane 121.

FIG. 14B depicts the arrangement where the bone cement 1444 is located on the outer periphery of the ring electrode 1459. Here, nonconductive bone cement 1444 is located such that the electrode 1459 can be placed directly against the outer periphery of the round window 121. As seen, lead 711 extends to lead 710 through the bone cement 1420, which can also be nonconductive bone cement.

Still, in an alternate embodiment, conducted bone cement can be utilized between the bottom of the ring electrode 1459 and the round window 121. In this manner, the round window will be permitted to move upward and downward substantially unrestrained. The lead 711 can be sufficiently flexible rather wise configured to move with the movement of the electrode so as to reduce or otherwise eliminate any resistance to movement associated with the electrode and/or the lead.

In view of the above, it can be seen that in at least some exemplary embodiments, the electrical current from the electrode flows across the round window first before entering bone, or otherwise entering the interior cochlea for that matter. This as opposed to the arrangement of FIG. 8 for example, where, for example, the electrical current enters bone before reaching the interior of the cochlea or otherwise the perilymph in the cochlea.

It is noted that while the teachings detailed herein are directed towards application to the round window, in an alternate embodiment, the teachings herein can be applicable to attachment to the oval window. This can have utilitarian value with respect to treating a recipient for tinnitus or otherwise mitigating the effects of tinnitus where the person has lost the conductive hearing portion of his or her hearing system, and relies on, for example, bone conduction or a middle ear implant for hearing.

The various concepts above present opportunities to utilize bone cement management techniques so as to control the spatial locations that result in bone cement being present with respect to the application of the bone cement in a recipient. By way of example, some embodiments include containing the bone cement within a certain area so as to prevent the bone cement from flowing onto nearby structures, at least where there could be less than utilitarian value with respect to bone cement contacting the structures.

More specifically, embodiments include a structure that supports the use of bone cement to attach the electrode(s) or otherwise hold them to bone on the outside of the otic capsule for stimulation therapy of tinnitus and/or vestibular dysfunction.

The bone cement can be conductive or non-conductive. Different designs of the electrode are used as detailed herein. In the case of conductive bone cement, the cement can be involved and around the stimulation contact (the electrode). In the case of non-conductive bone cement, it can be utilitarian to maintain the cement separate from the stimulation contact. In both instances some physical structure can be utilized to assist with compelling fixation/maintaining contact of the electrode to the bone. In both cases it can be utilitarian to control the flow of the bone cement so that the cement is contained and prevented from flowing onto the ossicular chain or other important structure of the middle ear.

FIG. 15 presents an exemplary implementation of a concept which can be used with conductive cement. The structures 1540 at the base of the apparatus provide a mechanical key that the conductive cement envelopes to improve retention relative to that which would be otherwise the case. FIG. 15 depicts an apparatus which can correspond to assembly 118 detailed above. Here, there is a lead assembly 1515 which can correspond to the lead apparatus of assembly 118 above with respect to FIG. 2, or can be the component extending from the housing 526 to the electrodes 520 of the embodiment of FIG. 6 (element 520 can be replaced with that of FIG. 15 or the other structures associated therewith, just as element 520 can be replaced with the electrode(s) detailed herein). Or the lead 1515 can extend from element 330 with respect to the embodiment of FIG. 4 (elements 220 of FIG. 3 or FIG. 4 can be replaced with that of FIG. 15 or the other structures associated there with, just as element 220 can be replaced with the electrode's detailed herein).

(Briefly, it is noted that any embodiment herein can be combined with any other embodiment herein unless otherwise specified or unless the art does not enable such. Briefly, the various electrodes detailed herein can be mixed and matched with the other associated structures. For example, the combination of elements 710 and 720 can correspond to lead 118 above, and/or can correspond to the leads to the electrodes of FIGS. 3 and 4 and 6 above.)

Lead assembly 1515 extends to body 1510, which can be a hollow tube with the tapered and as shown. In this exemplary embodiment, body 1510 is electrically conductive. Wire(s) with in lead assembly 1515 (the lead assembly comprises a sheath in which one or more electrical leads are located) are electrically connected to the body 1510. The body 1510 constitutes an electrode. The inside 1530 of the electrode 1510 is hollow, concomitant with a tubular body. The bottom is open. At the base of the tube are loops 1540, which can be wire or can be more substantial structures, such as rigid or semirigid bodies established by bio compatible material, which can be a metal such as platinum or can be a nonconductive material. In an exemplary embodiment, loops 1540 provide mechanical keying of the electrode body to the cement, thus enhancing the securement of the electrode body to bone. That said, in some embodiments, the loops 1540 can be of an arrangement that will embed themselves into bone and help further secure the attachment of the electrode to bone. Here, four loops are provided, but more or less loops can be utilized in some embodiments. In an exemplary embodiment, the loops are metal U-shaped components that are welded to the outside of the body 1510, although in other embodiments, the loops extend through holes in the body, and the loops are interference fitted therein and/or welded to the body 1510.

To be clear, the structure shown in FIG. 15 could be metal or a combination of materials including polymers and metals. The structures to key to the cement could be many shapes and include features such as sharp spikes to provide a positive fixation to prevent the electrode from sliding sideways while the cement is injected and/or curing.

In an exemplary embodiment, the loops can be flexible. In an exemplary embodiment, the loops can be pressed inward towards the longitudinal axis of the body 1510, and then placed into a predrilled hole or the like, and the flexible nature of the loops will drive the loops away from the longitudinal axis, and thus enhance a friction force established between the wall of the hole into which they are located. When bone cement is pumped into the interior 1530 of the body 1510, the bone cement will then travel downward into the hole, and then commingle with the loops, further securing the electrode.

As can be seen, there is a port 1520. This port is an angled cylindrical tube that extends from the side of the body 1510. This port can be made of a different material than that of the body, such as silicon. The port 1520 can be a silicone tube. That said, the port can be the same material as the body (e.g., platinum, etc.). In an exemplary embodiment, the port 1520 is welded to the body. Still, as noted, in an alternate embodiment, the port 1520 is made of a different material, and may be nonconductive. The port could be interference fitted into a hole on the side of the body 1510. The port could be glued thereto. Moreover, such as in the case where the port is silicone, the port could be a septum like apparatus that can be pierced by a wide diameter lumen of a syringe so that bone cement contained in the syringe can be “pumped” into the interior 1530 through the septum, where the lumen has created a hole therethrough. Any device, system, and/or method that can enable a convenient apparatus to channel bone cement into the interior 1530 the body can be utilized in at least some exemplary embodiments.

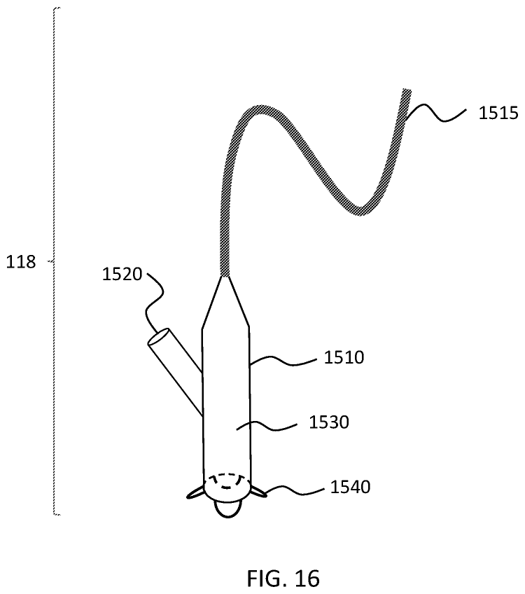

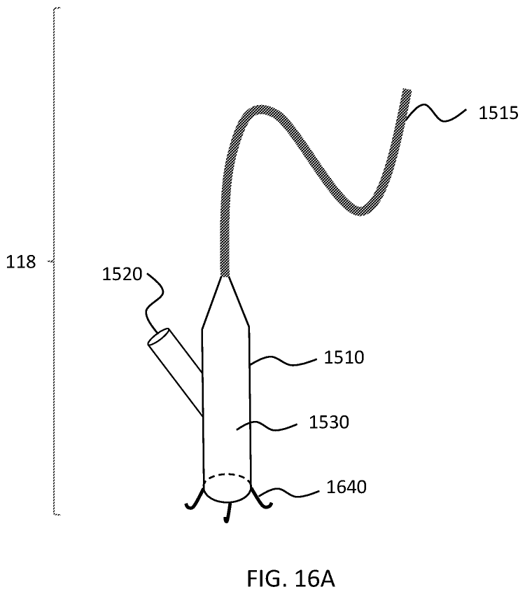

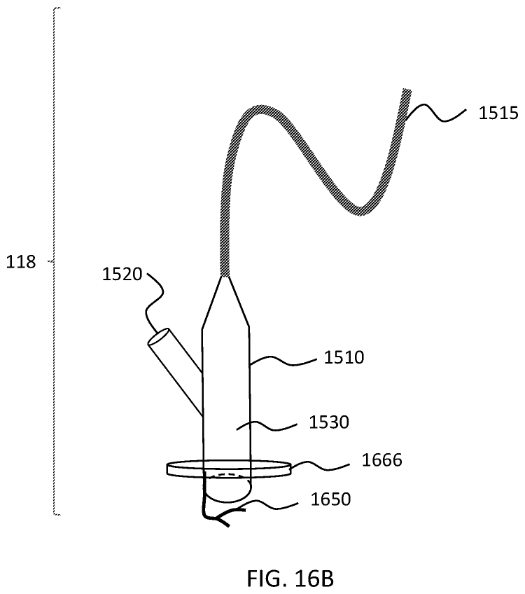

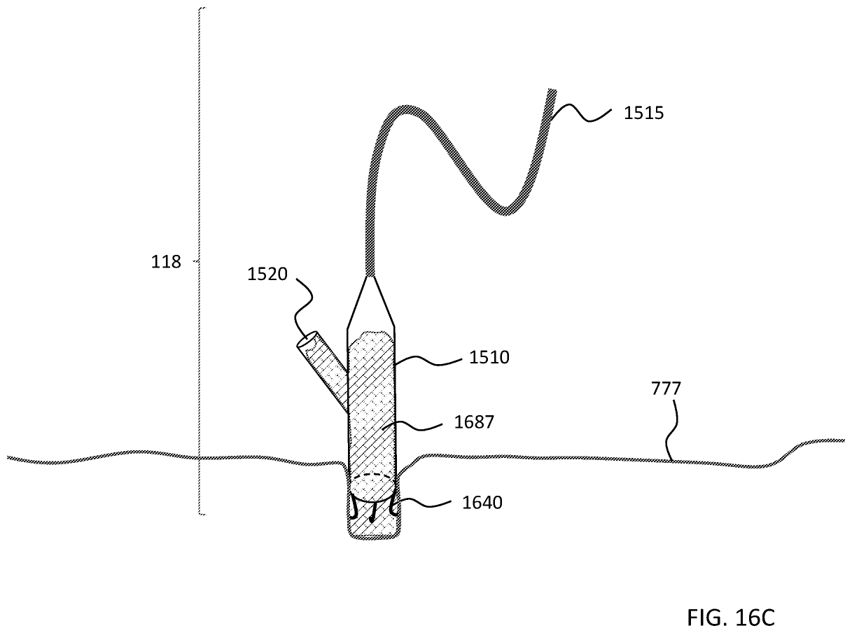

FIG. 16 provides an alternate embodiment where the loops 1540 (the apparatus that provides the keying) of the electrode body to the cement is angled downward so that the loops press into the bone. This can have utilitarian value with respect to further ensuring retention of the electrode body to the bone. In an exemplary embodiment, while the loops extend at a 90° angle relative to the longitudinal axis of the body 1510 in the embodiment of FIG. 16, in a relaxed state, the loops extend, in a relaxed state, at an angle of or more than 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260 degrees, or any value or range of values therebetween in 1° increments from the longitudinal axis of the body 1510. With respect to the angles greater than 180°, this can still have utilitarian value with respect to the loops that are located in the hole that is drilled, where the cement will commingle with the loops and that hole. It is further noted while flexible loops have been just described, in other embodiments, the loops are nonflexible. And to be clear, while loops have been disclosed as the embodiment here, in another embodiment, other arrangements can be utilized, such as the claw 1640 shaped arrangement of FIG. 16A. Also, a more distinct arrangement can be utilized which keys into the cement, but does not necessarily interface with the bone such as that shown in FIG. 16B where frame 1650 extends out from the interior 130, which frame 1650 is secured to the body 1510. The cement is commingled and surrounds the frame 1650 when they cement is channeled through the body 1510 and into the hole into which the body 1510 can be located or otherwise the hole that the frame 1650 extends.

The embodiment of FIGS. 15-16B can be utilized with conductive cement or utilized without conductive cement, provided that the body 1510 abuts the bone structure with a sufficient area to enable electrical conductivity. In an exemplary embodiment, by placing the body 1510 at least partially into the hole that is drilled in the bone, provided that there is contact between the outside of the body 1510 in the inside of the hole, there will be conductive the between the two. In an alternate embodiment, it could be sufficient that the bottom face of the body 1510 abuts the surface of the bone surrounding the hole into the bone. The cement can extend from inside the body 1510 to outside the body into the hole, and then adhere to the sides of the wall of the hole, the structure of the bone cement securing the electrode within the hole. In an exemplary embodiment, there can be components inside the interior 1530 that key to the cement that is located inside the body 1510, further adhering the cement to the electrode. An example of this is seen in FIG. 15, where barbs 1533 are adhered to the interior wall of the body 1510, where the bone cement envelop the barbs further securing the bone cement to the electrode body 1510.

FIG. 16B shows a conductive skirt 1666 that can be used to increase the contact area with the bone, thus further improving electrical conductivity therewith. In this exemplary embodiment, the body 1510 would be inserted into the hole into bone that is drilled, and the bottom of skirt 1666 would abut the bone. In an exemplary embodiment, and insulative material can be located on the outside upper portions of the skirt 1666. The skirt 1666 can be made out of platinum, and can be a disk through which a hole has been created, through which the body 1510 extends, such as in an interference fitted manner to control the location of the skirt relative to the longitudinal direction of the body.

FIG. 16C depicts an exemplary scenario of use, where bone cement 1687 has been injected into the interior of the tube 1510 through the port 1520. As can be seen, the bone cement 1687 extends from inside the tube 1510 to outside the tomb 1510 into the hole drilled into the bone 777. Also shown are hooks 1640 pressing against the side of the hole drilled into the bone 777. Upon curing of the bone cement 1687, the electrode will be secured into the bone.

In an exemplary scenario of use, the body 1510 can be held by a surgeon or the like and the tines/hooks 1640 (or loops, etc.), can be placed into the hole into the bone 777, one by one, and in the body 1510 can be canted/rotated and otherwise moved so that the other tines hooks be placed in the hole. Once they are all located in the hole, the tube 1510 can be pushed into the hole. In an exemplary embodiment, the tube 1510, or more accurately, the outside of the 215 10, establishes an interference fit or a slip fit or a snug fit or a clearance fit with the hole.

The bone cement effectively locks the electrode in place.

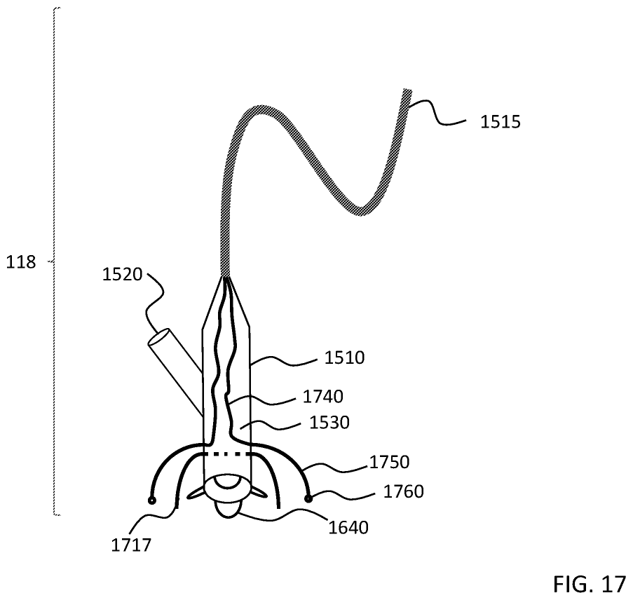

FIG. 17 presents an alternate exemplary embodiment can be utilized with nonconductive cement. In this exemplary embodiment, the cement is maintained within a silicone dome 1717 so that the bone cement does not flow around or otherwise towards the electrode contacts 1760, where the nonconductive bone cement could result in a high impedance scenario between the electrodes and the bone. More specifically, in this exemplary embodiment, the electrodes are ball electrodes 1760 located outside the interior 1530 of the tube 1510. Here, in this embodiment, the two 1510 can be made of a nonconductive material. Here, leads 1740 extend from lead assembly 1515 to outriggers 1750, which can be conductive material or can be material that houses a lead therein, which lead extends to the ball electrodes 1760. Further as can be seen, there is a shroud 1717. This shroud 1717 prevents the flow of bone cement or otherwise limits the flow of bone cement towards the ball electrodes 1760. Here, the outriggers 1750 are flexible and otherwise biased downward so that the ball electrodes are pushed against the outer surface of the bone in use.

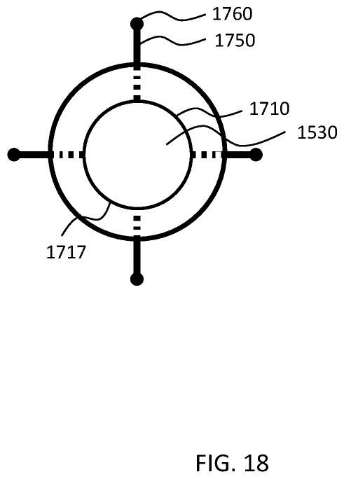

The electrodes 1760 contact an outer surface of the bone, as noted above, in an exemplary embodiment, the bottoms of the electrodes 760 are located at the same level as the mechanical loops on the end of the two 1510. These recess back from the level of the edge of the silicone dome so the dome 1717/shroud 1717 can be pressed on to the bone, and the slight compression resulting from further pressing until the tube 1510 and/or the electrodes contact the bone create/establish an adequate seal. The silicone dome/shroud (the dome/shroud can be made of other material in other embodiments, such as PEEK) contains the cement within the dome and prevents the cement from running around the electrode contacts 1760. FIG. 18 presents a bottom view of the embodiment of FIG. 17.

The electrical contacts radiating from the central body could be multiple separate electrodes allowing for bipolar, tripolar, quadripolar, or greater stimulation pattern (depending on the number of separate electrodes). This can be used with a non-conductive main body 1510.

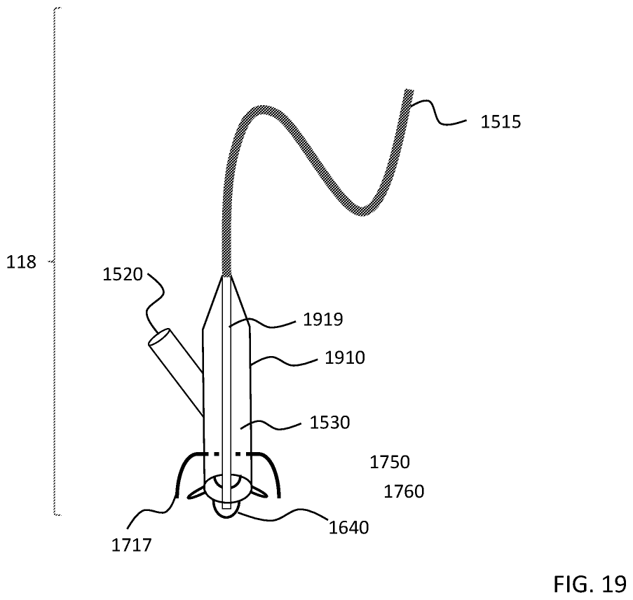

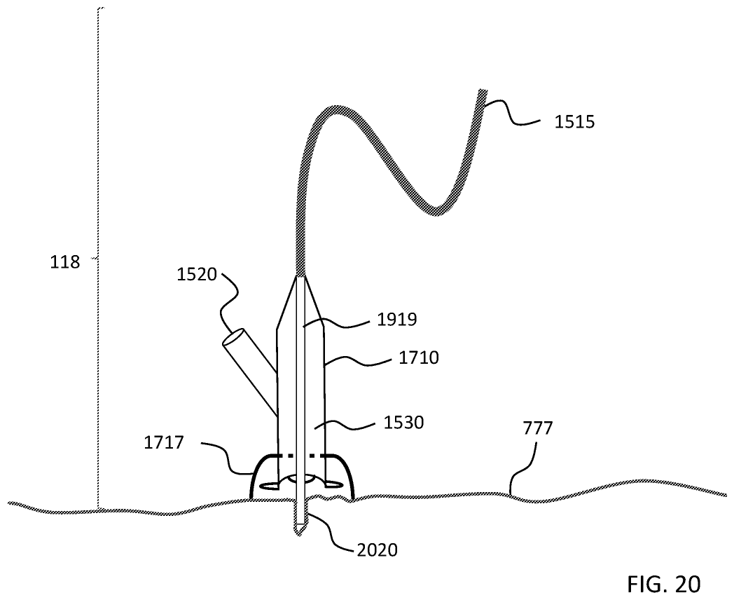

FIG. 19 presents another exemplary embodiment they can be used with either conductive or nonconductive cement. Here, there is a central electrode 1919 that can rest in a partial hole drilled in the bone. Here, dome 1717 is shown, but this may not necessarily be present in some embodiments (the loops may not be present in some embodiments). Here, body 1910 can be a nonconductive hollow tube/shield that contains bone cement and prevents or otherwise limits current leakage. Electrode 1910 can be a metallic electrode, and can be made of platinum or platinum-iridium or any other alloy or metal substance that can enable the teachings detailed herein, as is the case with respect to the other electrodes detailed herein. Here, the electrode 1919 can be designed to contact bone in general, and to contact the surface of a hole drilled into bone in particular. FIG. 20 presents an exemplary embodiment of this arrangement. Here, the electrode 1919 extends in a hole 2020. In this exemplary embodiment, the tube 1710 is held proud of the bone when the bone cement is cured. In an alternate embodiment, the hole can be drilled to accept as much of the electrode 1919 as needed until the bottom of the tube 1710 contacts the outer surface of the bone 777.

An exemplary embodiment is to have the metallic electrode 1919 housed within a non-conductive hollow structure that serves to contain the bone cement. This can, in some embodiments, electrically insulate the electrode apart from its tip which is held in contact with the bone (in an exemplary embodiment, in a well drilled in the bone), to prevent current leakage.

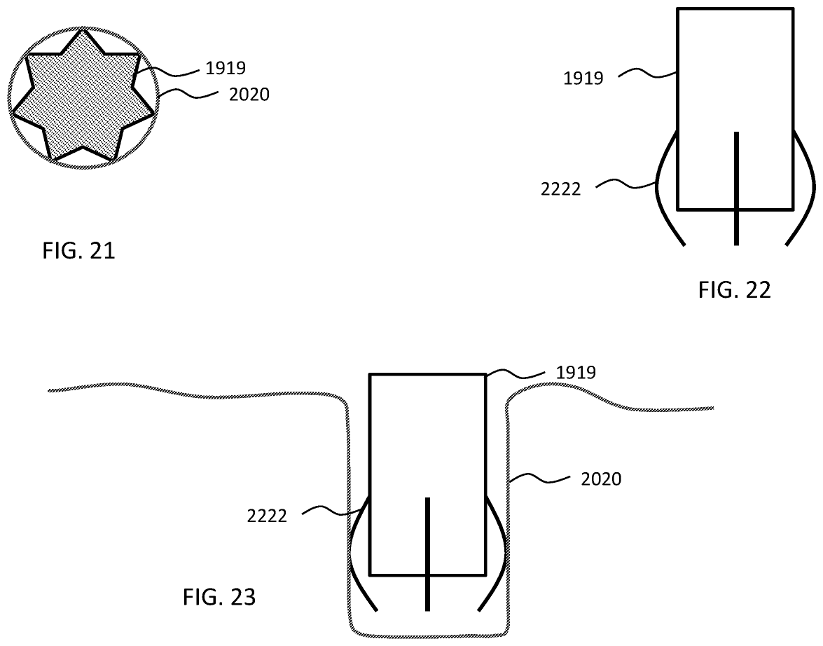

Another exemplary embodiment, of the arrangement of FIGS. 19 and 20 is shown in FIGS. 21-23. Here, the bone-end of the central electrode 1919 can be sculpted to have a mechanical fit to the bone. This can provide additional stability during injection and curing of the bone cement, and thereafter, beyond that which would otherwise be the case without this feature. This would also prevent bone cement invading the space around the electrode, thus keeping impedance low (or lower) between the electrode and bone in the scenario of non-conducting cement.

For example, as seen in FIG. 20, which shows the tip in a hole 2020 (looking out of the hole from the bottom of the hole). Here, the tip of electrode 1919 could be fluted to mesh with a hole drilled in the bone at a size just smaller than the outer diameter of the flutes (the outer extrapolated diameter). This provides an interference fit. The electrode is pressed into place prior to injecting the cement. The interference fit at least partially secures the electrode 1919 into the hole 2020. Then, bone cement is directed into the hole, at least partially filling the hole. The cured bone cement holds the electrode in the hole.

In another exemplary embodiment, the tip/end of the electrode 1919 can include tynes/splines designed to splay out as the electrode is pressed into place. FIG. 22 depicts an exemplary embodiment, and FIG. 23 depicts the result when placed into hole 2020. The tynes/splines push out and hold the electrode 1919 in place. The interference fit at least partially secures the electrode 1919 into the hole 2020. Then, bone cement is directed into the hole, at least partially filling the hole. The cured bone cement holds the electrode in the hole.

In another embodiment, the tip/end of the electrode 1919 can included a full thread (one or more turns) or a partial thread allowing a half-rotation of the electrode 1919 to create an initial hold in a suitably-sized hole in the bone. Moreover, the hole 2020 can be drilled/or excavated in the bone in a way that assists with initial fixation by having a wider base than the opening. This is seen in FIG. 23, which can be compared to FIG. 22.

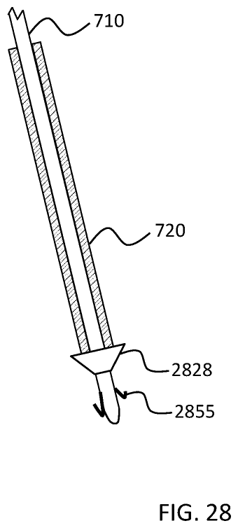

In view of the above, it can be seen that in some embodiments, the electrode includes keying structure (e.g., see below the arrangement of FIG. 28) at a location that interfaces with the bone and/or the securement material. In some embodiments, the electrode is one or more of fluted (see FIG. 21), threaded or splined at a location facing the bone.

In view of the above, in some embodiments, it can be seen that the electrode is an elongate electrode (which can be a cylinder or tube having a round cross-section, or can be a prismatic structure, or a box beam, or any configuration that can enable the teachings detailed herein) that extends inside the apparatus (in the tube 1910) that includes the arrangement that channels the uncured securement material to a location outside the arrangement (in FIG. 19, the tip of the electrode 1919 extends below the bottom portion of the tube 1910). Moreover, as can be seen in FIG. 19, the apparatus can include tynes, hooks and/or loops at a distal portion thereof configured to engage with bone and/or the securement material to enhance retention of the apparatus to the bone. Corollary to this is that the apparatus can include a king structure at a location at the distal end of the apparatus, such as those shown in FIGS. 19, 16C, 16B, etc.

A tapered tip can be used in some embodiments. The taper can fit like a wedge into the hole, and abut at the narrow area (potentially the opening). The bone cement can be pre-applied in the hole. A slit along the side of the tip can permit excess cement to ooze out of the hole as the tip displaces the cement.

In an exemplary embodiment, the lower most portion of the electrode extending into the hole extends a distance of greater than, less than or equal to 0.2, 0.3, 0.4, 0.5, 0.6, 0.7, 0.8, 0.9, 1, 1.25, 1.5, 1.75, 2, 2.5, 2.75, 3, 3.25, 3.5, 3.75, 4, 4.5, 5, 5.5, 6, 6.5, 7, 7.5, 8, 8.5, 9, 9.5, or 10 mm or more, or any value or range of values therebetween in 0.05 mm increments.

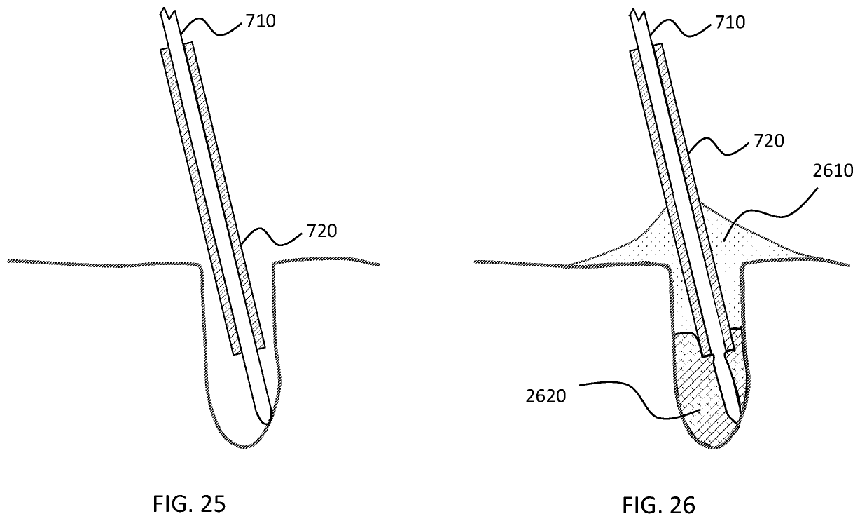

FIGS. 25-27 provide additional exemplary embodiments of some electrode placements. Here, the electrode is the lead 710. There is no discrete electrode per se. The electrode is the portion that extends from the insulator sheath 720, which can be a silicone sheath or some other bio compatible electrically insulated of body. In an exemplary embodiment, the electrode is placed into a hole in the bone, as seen in FIG. 25. Then, a conductive bone cement 2620 is placed in the hole at least partially filling the hole. Here, the level of the electrically conductive bone cement is below the level of the sheath, while in other embodiments, it may extend above the sheath. Then, in an exemplary embodiment, a nonconductive sealant, which may be a nonconductive bone cement 2610 is placed into the remainder the hole and extends proud of the hole in a manner akin to a shield volcano or the like, and the insulative material extends outward away from the hole on either side as seen.

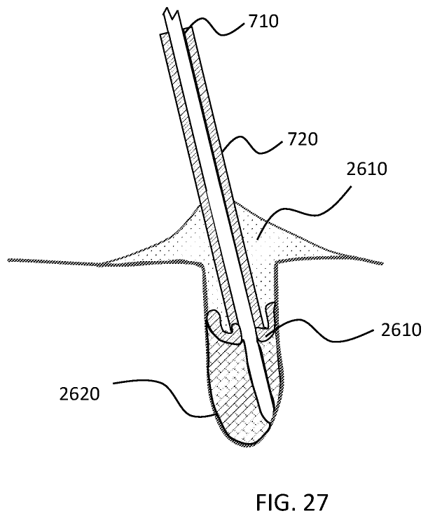

FIG. 27 presents an exemplary alternate embodiment of the embodiment of FIG. 26. Here, the sheath 720 splays outward at the end thereof, at section 2789. The sheath 720 thus can also form a plug. In the embodiment of FIG. 27, the sheath is depicted as two separate components that are bonded together, where the second component forms the portion that splays outward. In an exemplary embodiment, the sheath can be monolithic component, where the distal end can be of an arrangement that enables the splaying. While the nonconductive cement 2610 is shown, in some embodiments, this may not be utilized or otherwise may be dispensed with as the splayed sheath 720 provides a seal or otherwise provides a conductive the barrier between the exposed portion of the electrode and the ambient environment outside the hole. In an alternate embodiment, a plug, such as the plug detailed above can be located at the end of the sheath and/or around the sheath, which plug can be utilized to provide a conductive the seal or otherwise a high impedance barrier between the conductive bone cement 2620 and the ambient environment.

It is briefly noted that while in some embodiments, material 2620 is conductive and is a cement or other securement material, in other embodiments, the material 2620 is not a securement material. Instead, the material can be a conductive material that is easily flowable into the hole, but does not necessarily provide structural securement. That can be left to the material, 2610, which can also form is a plug as disclosed above. Any disclosure herein of placing a conductive securable material into the hole corresponds to a disclosure of placing a conductive non-securable material into the hole. Something that may otherwise hard and does not necessarily correspond to a securement material.

FIG. 28 presents an alternate exemplary embodiment where a plug 2828 is located at the end of the sheath 720 as can be seen. Plug 2828 can be made of silicone rubber or some other bio compatible material. The plug can be tapered as seen so as to provide a wedge fit into the bone. The plug 2828 can be resilient in some embodiments, while other embodiments can be somewhat rigid. In an exemplary embodiment, deformation of the bone can be relied upon to provide the seal between the conductive components and the ambient environment. Still, in some embodiments, it will be the plug 2828 that the forms. Other embodiments can use the plug arrangement and/or a variation thereof as will be applicable.

It is noted that while some embodiments depict areas where there is no bone cement between the bone and the cement, other embodiments include no such areas. Gaps may or may not be present with respect to the bone cement, depending on how the application process is executed. Embodiments include both.

Consistent with the teachings detailed herein, the electrode portion of the embodiment of FIG. 28 can have barbs or the like or otherwise can include components that enhance a keying effect the bone cement. As seen, barbs 2855 are located on the electrode portion 710. In an exemplary embodiment, these can be established by deforming a portion of the electrode, while in other embodiments, these barbs can be welded or otherwise adhered to the body of the electrode 710. When the electrode along with the barbs are placed into the bone cement, and the bone cement cures, increased resistance to the electrode pulling out from the hole will result.

While a wedge shaped plug is depicted in the above, in another embodiment, a disk or the like, or any other shape that can enable electrical insulation/sealing can be utilized in at least some exemplary embodiments. A disk can be deformable such that when the disk is inserted into the hole, the disk will flap upward at the outside diameter, somewhat as seen in FIG. 27.

In an exemplary embodiment, the teachings detailed herein can be utilized to achieve a relatively long term stable electrical impedance. In an exemplary embodiment, over any one or more of the aforementioned temporal periods detailed herein during which the electrode is secured or otherwise implanted in a recipient, the impedance will not vary more than an amount of 5, 10, 15, 20, 25, 30, 35, 40, 45, 50, 60, 70, 80, 90, 100, 125, 150, 175, or 200 percent, or any value or range of values therebetween in 1% increments from the initial impedance.

In view of the above, it can be seen that in at least some exemplary embodiments, an electrode can be inserted into a well drilled into a bone, such as the promontory bone, and the electrode can be utilized as a way to stimulate the cochlea, such as, by way of example only and not by way of limitation, for tinnitus suppression. In an exemplary embodiment, the hole can be drilled utilizing a standard drill or a routing bit/ball drill. This hole would not be a through hole, as noted above. The hole could be sized and dimensioned to establish an interference fit and/or slip fit and/or a clearance fit and/or a snug fit with the electrode in accordance with the teachings detailed herein.

In an exemplary embodiment, the partial holes that are drilled for tinnitus treatment or any other treatment that can have utilitarian value are drilled close to the semicircular canal, anywhere around the cochlea, around the atrium, etc. Any location that can be drilled that can have utilitarian value can be utilized in at least some exemplary embodiments.

In an exemplary embodiment, the stimulation can be executed to suppress or otherwise treat or otherwise mitigate the results for the effects of tinnitus while providing the recipients with residual hearing. In an exemplary embodiment, there is no impact on the residual hearing, while in other embodiments, there is relatively little impact on the residual hearing. As noted above, cost-benefit analysis can be made with respect to the potential for the impact on the residual hearing with respect to the benefits that results from the treatment of tinnitus or otherwise the mitigation or management of the effects thereof.

In an exemplary embodiment, at least 80, 85, 90, 92, 93, 94, 95, 96, 97, 98, 99, or 100% of a recipient's hearing is retained as residual hearing one month after implantation. The basis for the residual hearing can be a threshold hearing level across one or more frequencies, such as by way of example only and not by way of limitation, frequencies starting at 250 hz and increasing by 500 Hz up to about 8,000 kHz. in an exemplary embodiment, the residual hearing can be based on speech understanding utilizing standardized testing. In an exemplary embodiment, the aforementioned percentages are achieved with at least 80, 85, 90, 92, 93, 94, 95, 96, 97, 98, 99, or 100% of recipients.

In an exemplary embodiment, the aforementioned securement regimes for the electrode can have utilitarian value with respect to providing long-term electrical contact between the electrode and bone and/or while providing during this temporal period utilitarian electrical insulation from the middle in an exemplary embodiment, the long-term electrical contact can be for at least 5, 10, 15, 20, 25, 30, 35, 40, 45 or 50 years or more or can be any value or range of values therebetween in one year increments.

The teachings detailed herein can provide alternate methods of securing electrodes that avoid inserting an electrode in the cochlea. Indeed, in some exemplary embodiments of the teachings detailed herein, the recipient does not have an electrode in the cochlea, and the recipient is treated for tinnitus or otherwise the implant is utilized to mitigate or treat tinnitus. Conversely, the electrode can be implanted in a drill portion of the bone. As disclosed above, some exemplary embodiments can include the utilization of an electrically conductive bone cement, where the electrically conductive bone cement can be utilized to at least partially fill the hole drilled into the bone. Further, an electrically insulative bone cement can be utilized to establish a barrier between the electrically conductive components and the outside environment, such as the middle ear space. Also, in some exemplary embodiments, there is no stimulating electrode that is applied or otherwise placed on a surface of the skull, at least not one that is utilized for the treatment regimes detailed herein, such as tinnitus and/or epilepsy. This is distinguished from, for example, a return electrode, which might be so located. That said, in some embodiments, there is neither a stimulating electrode nor a return electrode that is located on a surface of the skull or otherwise the surface of a bone. Instead, one or more or all of the electrodes are located beneath the surface, such as in an artificial bone excavation or an artificial hole. Accordingly, in an exemplary embodiment, at least all of the read electrodes, in other embodiments all of the electrodes, are located beneath a surface of a skull and/or are not located on a surface of the skull (this latter scenario could be applicable to where the electrode is located on the round window—the electrode is not below the surface of the bone per se, but it is not on a surface of a bone either).

As noted above, in some embodiments, the electrode is placed in and/or on the promontory, which is a location of the basal turn of the cochlea, and is a bone that protects the cochlea. The electrodes can be placed in other locations with respect to any other bone that protects the cochlea or otherwise establishes the cochlea. In an exemplary embodiment, this can have utilitarian value with respect to stimulating the auditory nerve and/or the semicircular ducts with electricity from the electrode so as to stimulate the vestibular nerve. All of this can be done, in at least some exemplary embodiments, to treat tinnitus or otherwise mitigate the effects of tinnitus via the application of electrical current from the electrode. Still, as noted above, another location could be the round window or the oval window. Still further, in an exemplary embodiment, a partial cochleostomy might be made at a location adjacent the round or oval windows. In this embodiment, the cochleostomy does not extend through into the cochlea. The electrode could be placed into the resulting hole that results from the partial cochleostomy.

The above said, in other embodiments, the electrode can be placed at other locations, and in some embodiments, an excavation can be made of these other locations into which the electrode or another component according to the teachings detailed herein can be inserted.

Still, consistent with the teachings detailed above, other embodiments are applicable to treating epilepsy or otherwise managing the effects of epilepsy, and thus the electrodes would be placed in other locations, such as other locations about the cranial portion of the skull.

In view of the above, it can be seen that in at least some exemplary embodiments provide a bone embedded electrode (or a plurality of such) that provide utilitarian or otherwise at least adequate electrical environments that can enable the various treatments or mitigation therapies detailed herein.

In an exemplary embodiment, electrically conductive epoxies can be utilized, and any disclosure herein of a bone cement corresponds to an alternate disclosure of utilizing an epoxy, unless otherwise noted, or otherwise any other securement material that relies upon curing as used herein, and unless the art does not enable such. In an exemplary embodiment, there are resins filled with conductive particles or otherwise resins that have conductive particles dispersed therein. In an exemplary embodiment, the amounts of particles reaches or otherwise passes a percolation threshold where the density is high enough to allow tunneling or otherwise contact to make a conductive path. These conductive fillers can come in a range of materials metals to conductive polymers to the more exotic materials such as carbon nanotubes, graphene, etc. any arrangement that can enable the teachings detailed herein in a biocompatible mantle can be utilized in at least some exemplary embodiments.

Also, in some embodiments, medical grade electrically conductive silicone can be utilized. Any disclosure herein of bone cement or the like corresponds to an alternative disclosure of utilizing medical grade silicone, unless otherwise noted, and unless the art does not enable such.

An exemplary cement that can be used in some embodiments, or cements similar thereto that can be used, is disclosed in Australian patent document AU2008230047A1. Disclosure presents a bone cement that can be heated through RF induction to facilitate curing. While this disclosure discloses ferromagnetic particles, in other embodiments, the use of platinum or the like or platinum-iridium particles could instead be used. Alternatively, and/or in addition to this, the teachings of U.S. Pat. No. 9,610,110, to Csaba Trukai, et al, published on Apr. 4, 2017, can be utilized with respect to a bone cement. Furthermore, electrically conductive materials that are recognized as suitable for tissue engineering can be utilized such as those disclosed in the paper Electrically Conductive Materials: Opportunities and Challenges in Tissue Engineering, published in “biomolecules,” published online on Sep. 4, 2019, by Azadeh Saberi et al. providing that such enables use in accordance with the teachings detailed herein. In some other embodiments, materials that utilize polymethyl methacrylate in conjunction with electrically conductive materials that can be added as filler to the polymer can be used providing that such can be utilized in a biocompatible manner.

In view of the above, it can be seen that in some exemplary embodiments, there is an apparatus, such as a tinnitus mitigation and/or treatment system and/or epilepsy treatment and/or mitigation system, that comprises an electrode, wherein the electrode is at least one of directly or indirectly fixed to an otic capsule or tissue associated with the otic capsule of a human (e.g., the round window) at least in part with cured securement material. The cured securement material can be the conductive or nonconductive bone cement's detailed herein, the epoxies, or other materials disclosed herein that cure so as to affect the securement, or any other material that can have utilitarian value with respect to accomplishing the teachings detailed herein. Note that a cured securement material is different from, for example, mechanical fixture, such as a screw or the like, where the mechanical fixture does not cure to affect the securement. Consistent with the teachings detailed above, the electrode is fixed to the promontory of a cochlea of the human. By “fixed,” this does not mean that the fixation arrangement must be directly on the electrode. It can be enough that the fixation arrangement holds the electrode against the promontory. By way of example only and not by way of limitation, the cured material can be located about the nonconductive sheath and away from the conductive components of the electrode. Provided that the electrode is in electrical conductive attachment with the promontory, it is thus fixed to the promontory. That said, in an alternate embodiment, such as where the conductive bone cement is utilized, where the conductive bone cement completely envelops the electrode, if the conductive bone cement is located against the promontory, that two results in an electrode that is fixed to the promontory. Thus, in some embodiments, the cured securement material is electrically conductive, and the securement material is at least partially interposed between the electrode and the tissue of the otic capsule. In some embodiments where the securement material is bone cement the bone cement completely surrounds an outer periphery of the electrode from a plan view of the electrode.

Consistent with the teachings detailed above, the electrode of the aforementioned apparatus can be located in an artificial hole in the otic capsule. Conversely, in alternate embodiments, the electrode is in direct or indirect contact with a round window of a cochlea of the human. With respect to the latter scenario, it could be that some form of conductive securement material is located between the electrode and the round window. With respect to the former scenario, it can be that detailed above where the overall arrangement presses or otherwise holds the electrode against the round window.

Further, as noted above, the apparatus can include a tinnitus treatment and/or mitigation stimulator in electrical communication with the electrode. This can be any stimulation device that can enable the treatment and/or mitigation by providing an electrical signal to the electrode. In an exemplary embodiment, that stimulator is also implantable. That said, in an alternate embodiment, there is no implanted stimulator per se. Instead, the implanted inductance coil or the like receives power transcutaneously from an external unit, and the current that is generated in the coil is then transferred directly or indirectly to the electrode.

In some embodiments, the apparatus includes a keying structure, such as that of FIGS. 13, 13A, 15, 16, 16A, 16B, etc., keyed into the cured securement material, enhancing fixation of the apparatus to tissue of the human relative to that which would be the case in the absence of the keying structure. In an exemplary embodiment, a removal force that is required to pull the electrode from the secure location after curing is at least 30, 50, 70, 90, 100, 125, 150, 175, 200, 225, 250, 275 or 300 percent or more or any value or range of values therebetween in 1% increments more than that which would be the case in the absence of the keying. In an exemplary embodiment, any of the additional retention features detailed herein result in such performance relative to that which would be the case in the absence of those additional retention features (e.g., only surface to surface contact with bone cement is used).

In an exemplary embodiment, the apparatus includes splines and/or tynes and/or spikes that enhance attachment of the apparatus to tissue relative to that which would otherwise be the case. Again, these enhancement features can result in the just detailed performance values.

With respect to the embodiments associated with the electrode in contact with the round window of the cochlea, in at least some exemplary embodiments, the electrode is spring-loaded against the round window the cochlea, such as that seen in the embodiment of FIG. 14 and FIG. 14A. In an exemplary embodiment, the electrode is a nonpenetrating electrode against soft tissue of the inner ear, such as the electrode of FIG. 14A.

In some embodiments, the apparatuses detailed herein or in general comprise An apparatus, comprise an electrode. The electrode can be the tube 1510, for example. Further, the apparatus includes an arrangement that channels an uncured securement material, applied under pressure into the arrangement, to desired locations. Here, that arrangement can be at least in part the interior 1530 of the tube 1510. In an exemplary embodiment of this exemplary embodiment, the apparatus is an implantable apparatus implantable in a human recipient attachable to bone of the human recipient.

In some embodiments, the apparatus is configured to control the flow of the uncured securement material so that the uncured securement material is contained and prevented from flowing onto an ossicular chain of the human recipient and/or or other important structure of the middle and/or inner ear. Such control can be achieved via the tube 1510, and for example, placing the tube into an artificial hole/partial hole into bone, or with the above detailed shroud, or any other control techniques that can have utilitarian value with respect to achieving this feature. To be clear, in an exemplary method of implantation of this apparatus, the apparatus will be primarily located in the middle ear cavity or otherwise the location of attachment will be accessed through the middle ear cavity. Accordingly, the uncured securement material may flow in an undesired matter towards tissue, such as the ossicles, etc., where such flowing is not desired. In this exemplary embodiment, the entire flow management/deleterious flow prevention actions can be executed utilizing in its entirety the apparatus according to the teachings detailed herein, at least after the uncured material flows from the application device, such as a syringe or the like. That is, in an exemplary embodiment, no temporary flow management components are utilized in at least some exemplary methods of implantation of the apparatuses detailed herein. That said, in a scenario where such management components might have been used, the components have little to no effect on the overall management/control of the flow. By way of example only and not by way of limitation, a mechanic might hold an oil rag against a portion of an engine, but if the flow of oil is controlled in a given manner, even though the rag is used, the rag has no effect if the oil does not contact the rack. That is, the rag is utilized in an abundance of caution for example.

Consistent with the teachings detailed above, it is to be understood that any action detailed herein that would be utilized or otherwise required to implement any of the teachings detailed herein corresponds to a disclosure of a method of doing so. In this regard, embodiments according to the teachings detailed herein include methods. In one exemplary embodiment, there is a method comprising treating a neurological ailment and/or mitigating effects of the neurological ailment of a human with an electrode. The neurological ailment can be epilepsy. The neurological ailment can be tinnitus by way of example. These are things related to the neural network, where tinnitus, for example, is the stimulation of neurons that results in a hearing percept, and unwanted hearing percept. Thus, tinnitus is a neurological ailment as that phrase is utilized herein. The treatment can be executed at least one of applying electrical current to an electrode at least one of (1) directly or indirectly fixed to tissue of a recipient with cured electrically conductive securement material, (2) located in a partial artificial hole (as opposed to a through hole) in bone of a skull or (3) directly or indirectly fixed to tissue of a recipient with cured securement material, wherein the electrode is in electrically conductive communication with soft tissue in the head of the human. With respect to the latter, this can correspond to the embodiments of FIGS. 14 and 14A, for example. That would be indirect fixation. In an exemplary embodiment where an adhesive is utilized to hold the electrode to the membrane 121, where the adhesive would be located between the electrode 1410 and the membrane, that would be direct fixation. With respect to the scenario of locating the electrode in a partial artificial hole, that can correspond to the teachings detailed above for example with respect to FIG. 16 C or FIG. 23, etc. with respect to the first scenario, that could cover the arrangement of FIG. 8, which would be indirect fixation, or the arrangement of FIG. 10B, which is direct fixation.

Another way of considering rather wise evaluating features associated with the electrode relative to tissue of the recipient is by considering whether or not the electrode is in direct or indirect contact with tissue. The arrangement of FIG. 10B would be direct contact, whereas the arrangement of FIG. 8 would be indirect contact.

In an exemplary embodiment where the neurological ailment is tinnitus, and the electrode is located in a partial artificial hole in the otic capsule, the electrode can be in direct contact or in indirect contact with the bone of the capsule, FIG. 10B showing direct contact, and FIG. 13A showing indirect contact.

In an exemplary embodiment, the method of treating the neurological ailment includes conducting an electrical current from the electrode into cured bone cement, and from bone cement into tissue of a human, thereby treating and/or mitigating the neurological ailment. In this embodiment, although in other embodiments, irrespective of whether or not the current travels through cured bone cement, at least directly from the electrode to the tissue (as opposed to the embodiments where the current can travel directly from the electrode to the tissue because the electrode is directly against the tissue), the current is applied in a manner that will stimulate a certain portion of the cochlea out to evoke a neurological reaction for certain nerves that tonotopic correspond to certain frequency and/or a certain frequency range as opposed to other nerves. In an exemplary embodiment, the current is applied in a manner that will result in the overall auditory nerve being stimulated at a certain frequency and/or a certain frequency range as opposed to other frequencies. In an exemplary embodiment, there will be no stimulation and other frequencies. In an exemplary embodiment, the current that is applied can be controlled with respect to its amplitude or otherwise the magnitude of the resulting stimulation, and can be adjusted from recipient to recipient to account for different specific physiological conditions of that recipient.

In an exemplary embodiment, the frequency ranges constitute one, two, three, four or more specific frequency ranges which may or may not overlap each other, where the various ranges can span 50 or 100, 150, 200, 250, 300, 350, 400, 450, 500, 550, 600, 700, 800, 900 or 1000 Hz or any value or range of values there between in 1 Hz increments. Again, the frequent skis can be specific exact frequencies within plus or minus five, or four, or three, or two, or one Hz.

In any event, in at least some exemplary embodiments, the current is applied at an alternating frequency, or at least an alternating carrier frequency. This as opposed to direct current. That said, in an alternate embodiment, irrespective of whether or not it is tinnitus treatment/mitigation or epilepsy treatment or mitigation, the current could be applied with direct current. In some embodiments, the magnitude of the current and/or the frequency of the current can be varied one, two, three, four, five, six, seven, eight, nine, 10, 15, 20, 25, 30, 35, 40, 45, 50, 55,60 times or more or any value or range values therebetween in one increment within a temporal time period, such as a time period lasting one, two, three, four, five, six, seven, eight, nine, 10, 15, 20, 25, 30, 35, 40, 45, 50, 55, 60 seconds or minutes or any value or range values therebetween in 1 increment. As noted above, with respect to the application of the teachings detailed herein to treat or otherwise mitigate tinnitus, the electrode can be pressed or otherwise held against a round window of a cochlea, and the electrode can be indirectly fixed (or directly fixed, where the electrode actually contacts the bone—in an exemplary embodiment, this can have utilitarian value with respect to providing two routes of electrical conductive of the two inside the cochlea) to bone of the otic capsule (round window niche) with the cured securement material, indirectly fixing the electrode to the round window.

With respect to the teachings detailed above, such as those where an artificial partial hole is excavated in the bone, the electrode can be located outside the cochlea, but at least partially beneath an extrapolated surface of the otic capsule. The extrapolated surface would be, for example, with respect to FIG. 13A, a hypothetical surface that extends slightly above the bottom portion of the plug 1185. The extrapolated surface is the surface that would otherwise be present but for the excavation of the hole. The extrapolated surface could be considered a surface that extends from one side of the hole to the other side of the hole at the top most portion of the hole.

Consistent with the teachings detailed above, where the neurological ailment is tinnitus, the treatment and/or mitigation can be executed such that at least 80, 90, 95 or 100% of the human's hearing is retained during the treatment and/or mitigation. Specific values and qualitative and quantitative features associated therewith have been described above and the reader is referenced thereto. With respect to the phrase “during treatment/mitigation” it is meant the temporal period where the implanted system is utilized to stimulate the tissue. In another exemplary embodiment, the treatment is executed such that the aforementioned percentages of the human's hearing is retained after implantation of the system, for at least 1, 2, 3, 4, 5, 6, 7, 8, 9 or 10 years or any value or range of values therebetween in one month increments.

It is briefly noted that any disclosure herein corresponding to treatment corresponds to an alternate disclosure related to mitigation and vice versa.

Embodiments also include methods of electrode and/or system implantation. For example, there is a method, comprising placing an electrode against tissue of an inner ear of a human (the otic capsule or the round window, for example). The method further includes applying uncured securement material to bone of the human to hold the electrode against the tissue of the inner ear of the human. In accordance with at least some exemplary embodiments, the method includes channeling the uncured securement material to the bone using a part of an assembly of which the electrode is apart. This can be the port(s) and/or the tube of the embodiment of FIG. 15. In some embodiments, the uncured securement material is electrically conductive when cured, while in other embodiments, it is not electrically conductive when cured. To be clear, in some embodiments, the teachings detailed herein include curing the material. In some exemplary embodiments, this can be executed utilizing the application of heat and/or RF energy or other types of energy and/or vibrational energy so as to speed the curing or otherwise enable the curing. In some embodiments, the securement material is not electrically conductive when cured. In some embodiments, the tissue is bone of an otic capsule of a human, and in some embodiments, the tissue is a round window of the cochlea of the human.

Embodiments of this method further includes the action of drilling and/or excavating a partial hole in the bone of the recipient, and placing the electrode in the hole. These methods can also include filling, at least partially, the hole with a curable securement material that is electrically conductive when cured. This securement material holds the electrode within the hole.

It is briefly noted that the curable materials detailed herein can enhance or otherwise increase the retention of the electrode relative to that which would otherwise be the case by an amount of at least 50, 75, 100, 150, 200, 250, 300, 350, 400, 450, 500, 600, 700, 800, 900 or 1000 percent or more or any value or range of values therebetween in 1% increments with respect to a pulling force directly away from the bone relative to that which would otherwise be the case in the absence of the retention material.

In at least some exemplary embodiments, as noted above, the electrode that is utilized as part of the method is part of the tinnitus management and/or treatment assembly, and this method further comprises implanting the assembly in a human. This can be done in a manner concomitant with that associated with the implantation of the implantable component of a cochlear implant as would be readily understood in the art. While noted above, in some embodiments, the system includes a stimulator device, which stimulator device can be akin to that utilized in a cochlear implant, in other embodiments, there is no stimulator device per se, an electrical current flows directly or indirectly from a receiver RF coil to the electrode.

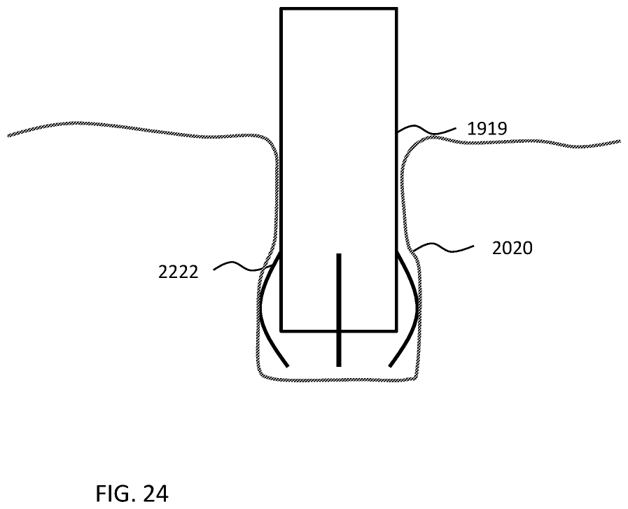

Consistent with the embodiment of FIG. 24, the action of drilling and/or excavating a partial hole in the bone of the recipient can result in a compound hole (e.g., where the lower portion is wider than the upper portion—a compound hole does not include the bottom feature resulting from the angled portion of a drill bit). In some exemplary embodiments, the action of placing the electrode into the hole is such that the such that the compound features of the hole at least partially retain the electrode in the hole. This is the case with respect to the embodiment of FIG. 24, for example.

In an exemplary embodiment, the components 2222 of FIG. 24 can be electrically conductive, while in other embodiments, there are not electrically conductive. The embodiment of FIG. 24 does not show the bone cement that would be located in the hole 2020.

With respect to the embodiment of FIG. 24, the electrode does not contact directly the wall of the hole. In this regard, in an exemplary embodiment, there can be a method of at least partially placing the electrode into the partial hole (the arrangement of FIG. 24) and placing the uncured securement material in the hole, wherein the material directly interfaces with the bone and the electrode. With respect to this exemplary method, the action of placing the uncured securement material in the hole can be executed before or after the action of at least partially placing the electrode into the partial hole. In this regard, unless otherwise specified, any method action detailed herein, regardless of the order of presentation, can be a method action that occurs before or after another method action, again regardless of the order of presentation, providing that the art enable such.

Consistent with at least some of the teachings detailed above, the action of inserting the electrode in the hole can include interference fitting and/or partially screwing the electrode into the hole, or any other technique detailed herein.

It is noted that any disclosure of a device and/or system herein corresponds to a disclosure of a method of utilizing such device and/or system. It is further noted that any disclosure of a device and/or system herein corresponds to a disclosure of a method of manufacturing such device and/or system. It is further noted that any disclosure of a method action detailed herein corresponds to a disclosure of a device and/or system for executing that method action/a device and/or system having such functionality corresponding to the method action. It is also noted that any disclosure of a functionality of a device herein corresponds to a method including a method action corresponding to such functionality. Also, any disclosure of any manufacturing methods detailed herein corresponds to a disclosure of a device and/or system resulting from such manufacturing methods and/or a disclosure of a method of utilizing the resulting device and/or system.

Unless otherwise specified or otherwise not enabled by the art, any one or more teachings detailed herein with respect to one embodiment can be combined with one or more teachings of any other teaching detailed herein with respect to other embodiments. Also unless otherwise specified or otherwise not enabled by the art, any one or more teachings detailed herein with respect to one embodiment can be explicitly excluded from use with one or more other features of other embodiments of any other embodiment herein with respect to other embodiments.

While various embodiments have been described above, it should be understood that they have been presented by way of example only, and not limitation. It will be apparent to persons skilled in the relevant art that various changes in form and detail can be made therein without departing from the spirit and scope of the invention. Thus, the breadth and scope of the present invention should not be limited by any of the above-described exemplary embodiments, but should be defined only in accordance with the following claims and their equivalents.

1. An apparatus, comprising:
an electrode, wherein the electrode is at least one of directly or indirectly fixed to an otic capsule or tissue associated with the otic capsule of a human at least in part with cured securement material.

2. The apparatus of claim 1, wherein:
the electrode is fixed to the promontory of a cochlea of the human.

3. The apparatus of claim 1, wherein:
the electrode is located in an artificial hole in the otic capsule.

4. The apparatus of claim 1, wherein:
the electrode is in direct or indirect contact with a round window of a cochlea of the human.

5. (canceled)

6. The apparatus of claim 1, wherein:
the cured securement material is electrically conductive, and the securement material is at least partially interposed between the electrode and the tissue of the otic capsule.

7-10. (canceled)

11. The apparatus of claim 1, wherein:
the electrode is a conductive hollow structure; and
the hollow structure contains at least some of the cured securement material, wherein the securement material is bone cement.

12. (canceled)

13. The apparatus of claim 1, wherein:
the apparatus includes a hollow structure; and
the apparatus includes a port for the injection of uncured securement material into the hollow structure.

14. The apparatus of claim 1, wherein:
the apparatus includes a shroud configured to at least limit a flow of uncured securement material prior to curing.

15. (canceled)

16. The apparatus of claim 1, wherein:
the securement material is cured bone cement and the cured bone cement is conductive.

17-31. (canceled)

32. An apparatus, comprising:
an electrode, wherein
the apparatus includes an arrangement that channels an uncured securement material, applied under pressure into the arrangement, to desired locations, and
the apparatus is an implantable apparatus implantable in a human recipient attachable to bone of the human recipient.

33. (canceled)

34. The apparatus of claim 32, wherein:
the apparatus includes a shroud that extends about the channel, which shroud contains the uncured securement material therein.

35. The apparatus of claim 32, wherein:
the apparatus includes a support structure supporting the electrode; and
the support structure includes at least one outrigger that supports the electrode away from the arrangement that channels the uncured securement material.

36. The apparatus of claim 32, wherein:
the electrode is an elongate electrode that extends inside the apparatus that includes the arrangement that channels the uncured securement material to a location outside the arrangement.

37. The apparatus of claim 32, wherein:
the apparatus includes tynes, hooks and/or loops at a distal portion thereof configured to engage with bone and/or the securement material to enhance retention of the apparatus to the bone.

38. The apparatus of claim 32, wherein:
the electrode includes keying structure at a location that interfaces with the bone and/or the securement material.

39. The apparatus of claim 32, wherein:
the electrode is one or more of fluted, threaded or splined at a location facing the bone.

40. A method, comprising:
placing an electrode against tissue of an inner ear of a human; and
applying uncured securement material to bone of the human to hold the electrode against the tissue of the inner ear of the human.

41. The method of claim 40, further comprising:
channeling the uncured securement material to the bone using a part of an assembly of which the electrode is apart.

42. The method of claim 40, wherein:
the uncured securement material is electrically conductive when cured.

43. The method of claim 40, wherein:
the uncured securement material is bone cement that is electrically conductive when cured.

44. The method of claim 40, wherein:
the tissue is bone of an otic capsule of the human.

45. The method of claim 40, wherein:
the tissue is a round window of a cochlea of the human.

46-49. (canceled)
